(19) United States
(12) Patent Application Publication
(10) Pub. No.: US 2006/0099170 A1
(43) Pub. Date: May 11, 2006

(54) Compositions for inducing increased levels of beta-chemokines and methods of use therefor

(76) Inventors: Robert R Redfield, Baltimore, MD (US); Anthony Amoroso, Baltimore, MD (US); Charles E Davis JR., Laurel, MD (US); Alonso Heredia, Washington, DC (US)

Correspondence Address: INTELLECTUAL PROPERTY / TECHNOLOGY LAW, PO BOX 14329, RESEARCH TRIANGLE PARK, NC, 27709, US

(21) Appl. No.: 10/527,904

(22) PCT Filed: Sep. 12, 2003
(86) PCT No.: PCT/US03/28697

§ 371 Date: Jul. 7, 2005

Related U.S. Application Data
(60) Provisional application No. 60/410,714, filed on Sep. 13, 2002.

(51) Int. Cl. A61K 38/19; A61K 38/12; A61K 31/7076; A61K 39/395; A61K 31/551; A61K 31/522; A61K 31/355; A61K 31/19

(52) U.S. Cl. 424/85.1; 514/45; 514/49; 514/220; 514/263.32; 514/291; 514/458; 514/557; 514/575; 424/145.1

(57) ABSTRACT

The present invention relates to compositions and methods for inducing increased levels and availability of β-chemokines by administering to a subject at least one G1 phase arresting compound, wherein the increased levels and availability of β-chemokines block chemokine/viral receptors thereby preventing or treating viral infections.

### BACKGROUND OF THE INVENTION

1. Field of the Invention

The present invention generally relates to increased production of β-chemokines, and more particularly, to compositions comprising at least one G1 phase arresting agent thereby resulting in increased levels and availability of β-chemokines to prevent or treat viral infections and viral related cancers, such as HIV infections and related HIV cancers.

2. Background of the Related Art

The human immunodeficiency virus (HIV) has been implicated as the primary cause of the slowly degenerative immune system disease termed acquired immune deficiency syndrome (AIDS). There are at least two distinct types of HIV: HIV-1 and HIV-2. In humans, HIV replication occurs prominently in CD4 T lymphocyte populations, and HIV infection leads to depletion of this cell type and eventually to immune incompetence, opportunistic infections, neurological dysfunctions, neoplastic growth, and ultimately death.

HIV is a member of the lentivirus family of retroviruses. Retroviruses are small-enveloped viruses that contain a single-stranded RNA genome, and replicate via a DNA intermediate produced by a virally encoded reverse transcriptase, an RNA-dependent DNA polymerase.

The HIV viral particle comprises a viral core, composed in part of capsid proteins, together with the viral RNA genome and those enzymes required for early replicative events. Myristylated gag protein forms an outer shell around the viral core, which is, in turn, surrounded by a lipid membrane envelope derived from the infected cell membrane. The HIV envelope surface glycoproteins are synthesized as a single 160 kilodalton precursor protein, which is cleaved by a cellular protease during viral budding into two glycoproteins, gp41 and gp120. gp41 is a transmembrane glycoprotein and gp120 is an extracellular glycoprotein, which remains non-covalently associated with gp41, possibly in a trimeric or multimeric form.

HIV is targeted to CD4 cells because a CD4 cell surface protein (CD4) acts as the cellular receptor for the HIV-1 virus. Viral entry into cells is dependent upon gp120 binding the cellular CD4 receptor molecules, explaining HIV's tropism for CD4 cells, while gp41 anchors the envelope glycoprotein complex in the viral membrane. While these virus:cell interactions are necessary for infection, there is evidence that additional virus:cell interactions are also required.

HIV infection is pandemic and HIV-associated diseases represent a major world health problem. Although considerable effort is being put into the design of effective therapeutics, currently no curative anti-retroviral drugs against AIDS exist. The new treatment regimens for HIV-1 show that a combination of anti-HIV compounds, which target reverse transcriptase (RT), such as azidothymidine (AZT), lamivudine (3TC), dideoxyinosine (ddl), tenofovir, nevirapine, efavirenz, or anti-HIV compounds which target HIV protease such as saquinavir, nelfinavir, indinavir, amprenavir, and lopinavir. For example, impressive results have recently been obtained with a combination of AZT, 3TC and a protease inhibitor as well as AZT, 3TC, and Efavirenz have demonstrated potent antiviral activity. Unfortunately the development of viral resistance occurs in a significant number of treated patients. This combined with the development of anti-retroviral drug induced toxicity continues to limit the overall impact of current available treatments.

Moreover, long-term cytotoxic therapy may also lead to suppression of CD8⁺ T cells, which are essential to the control of HIV, via killer cell activity and by the release of suppressive factors, notably the chemokines.

Chemokines are a family of small cytokines that are released in response to infection together with other inflammatory cytokines. Chemokines are multiple mediators, but were first studied as inducers of chemotaxis of specific leukocytes. Further studies have revealed that chemokines also stimulate lymphocyte development, angiogenesis, degranulation of granulocytes, respiratory bursts and the release of lysosomal enzymes in monocytes.

Chemokines are divided into four different subfamilies, according to the position of the first two cysteines in their primary sequence: the α-chemokine subclass bears a CXC-motif, where the two cysteines are separated by one amino acid; the β-chemokines contain a CC motif; the γ-subclass lacks one cysteine residue; and in Δ-chemokines, or CX₃C subclass, the two cysteines are separated by three amino-acids. These cysteine residues form disulfide bridges with two other cysteines located further downstream in the primary sequence, thus stabilizing the tertiary structure of these chemokines.

Recently, chemokines produced by CD8⁺ T cells have been implicated in suppression of HIV infection. The chemokines RANTES, MIP-1α and MIP-1β, which are secreted by CD8⁺ T cells, were shown to suppress HIV-1 p24 antigen production in cells infected with HIV-1 or HIV-2 isolates in vitro (Cocchi, et al., 1995). However, levels of available chemokines are limited and the effectiveness of introducing exogenous chemokines is still in question because of the short serum half-life of exogenously administered chemokines.

Chemokine receptors are designated CXCR followed by a number when binding α-chemokines and CCR followed by a number when binding β-chemokines. The importance of CCR5 for initial transmission of HIV-1 is highlighted by the fact that individuals lacking expression of CCR5 (the CCR5-Δ32 homozygous genotype) are usually resistant to infection (Liu, et al., 1996). In addition, recent studies show that CCR5 cell-surface density correlates with disease progression in infected individuals (Lin, et al., 2002).

The natural ligands of CCR5 that include the β-chemokines macrophage inflammatory protein (MIP)-1α, MIP-1β, and RANTES, inhibit entry of CCR5 (R5) strains of HIV-1 in both lymphocytes and macrophages (Cocchi, et al., 1995). The inhibitory effect of β-chemokines is proposed to act through blocking of CCR5 as well as through down-regulation of the coreceptor from the cell surface. Inhibition of viral entry has also been achieved by blocking the binding of the viral gp120 to the CCR5 coreceptor by antagonist molecules such as TAK-779 (Baba, et al., 19990) or SCH-C (Strizki, et al., 2001). The β-chemokines RANTES (regulated on activation, normal T cell expressed and secreted), macrophage inflammatory protein-1α (MIP-1α), and MIP-1β are the natural ligands of CCR5, the main coreceptor of non-syncytium-inducing HIV-1, and have been shown to inhibit the in vitro infection of lymphocytes (Cocchi, et al., 1995).

The increase of RANTES, MIP-1α, and MIP-1β has been correlated with resistance to infection or a more favorable clinical prognosis, likely because of competition of the chemokines with HIV-1 for binding to CCR5 (Paxton, et al., 1996; Zagury, et al., 1998; Ferbas, et al., 2000; Cocchi, et al., 1995; and Adams, et al., 1997). However, natural levels and availability of chemokines are limited and as the effects of HIV infection increase the natural production of β-chemokines is decreased. Moreover, attempts to increase levels of chemokines have been centered on administration of chemokines directly to a subject by means of administering exogenous proteins directly to the subject. Depending on the mode of administration the amount of chemokines delivered is variable.

Thus, it would be advantageous to identify compounds that induce the increased levels of endogenous chemokines through pathways that modulate the activity of chemokines and increase the levels and availability thereof.

### SUMMARY OF THE INVENTION

The present invention relates to enhancing levels and availability of anti-HIV β-chemokines by manipulating the cell cycle in activated lymphocytes by administering a composition that prolongs the G1 phase of the cell cycle, thereby increasing production or available levels of β-chemokine by the activated lymphocytes, which, in turn reduces the effects of HW and related complications, such as related viral cancers.

In one aspect, the present invention provides for the modification of synthesis of known receptor ligands, such as chemokines, that alter the extracellular recognition of a receptor by an infectious agent, resulting in treatment of the disease or condition. Preferred receptors include: chemokine coreceptors, which mediate host cell uptake of viruses such as HIV. Examples of preferred receptor ligands include chemokines and receptor binding portions thereof. Particularly preferred chemokine ligands include MIP-1α, MIP-1β, and RANTES.

In another aspect, the present invention relates to enhancing levels of anti-HIV β-chemokines by manipulating the cell cycle in activated T cells by administering a composition that prolongs the G1 phase of the cell cycle, thereby increasing the overall β-chemokine production levels by activated T-cells, thereby inhibiting binding of HIV to β-chemokine receptors which, in turn, prevents or reduces replication of HIV.

In another aspect, the present invention relates to compositions that inhibit CCR5-mediated viral entry of HIV by increasing the number and availability of β-chemokine by exerting G1 cytostatic activity in mononuclear cells including, but not limited to T cells, activated T cells and macrophages.

In another aspect, the invention relates to a composition comprising a G1 cell cycle agent that delays entry of the S-phase in a mononuclear cell cycle thereby increasing the period of chemokine production in the G1 phase.

The G1 cell cycle agent may include any compound that arrests or prolongs the G1 phase in the cell cycle of mononuclear cells, for example, including but not limited to sodium butyrate, aphidicolin, hydroxyurea (HU), olomoucine, roscovitine, tocopherols, including alpha-tocopherol, beta-tocopherol, D-alpha-tocopherol, delta-tocopherol, gamma-tocopherol, tocotrienols, rapamycin (RAPA) and functional analogs thereof.

The compositions of the present invention may further comprise at least one antiviral agent. The antiviral agent may include any agent that inhibits entry into a cell or replication therein of an infectious virus, and specifically retroviruses, such as HIV viruses. The antiviral agents include, but are not limited to nucleoside RT inhibitors, CCR5 inhibitors/antagonists, viral entry inhibitors and their functional analogs.

Thus, in one aspect the compositions and methods of the present invention further comprise a therapeutically effective amount of at least one antiviral agent, including, but not limited to:

nucleoside RT inhibitors, such as Zidovudine (ZDV, AZT), Lamivudine (3TC), Stavudine (d4T), Didanosine (ddl), Zalcitabine (ddC), Abacavir (ABC), Emirivine (FTC), Tenofovir (TDF), Delaviradine (DLV), Efavirenz (EFV), Nevirapine (NVP), Fuzeon (T-20), Saquinavir (SQV), Ritonavir (RTV), Indinavir (IDV), Nelfinavir (NFV), Amprenavir (APV), Lopinavir (LPV), Atazanavir, Combivir (ZDV/3TC), Kaletra (RTV/LPV), Trizivir (ZDV/3TC/ABC);

CCR5 inhibitors/antagonists, such as SCH-C, SCH-D, PRO 140, TAK 779, TAK-220, RANTES analogs, AK602, UK-427, 857, monoclonal antibodies;

viral entry inhibitors, such as Fuzeon (T-20), NB-2, NB-64, T-649, T-1249, SCH-C, SCH-D, PRO 140, TAK 779, TAK-220, RANTES analogs, AK602, UK-427, 857; and functional analogs or equivalents thereof.

Another aspect of the present invention relates to a method to increase levels of anti-HIV β-chemokines, the method comprising:

administering a composition comprising an effective amount of a compound that arrests or prolongs the G1 phase of an activated T cell, thereby increasing production time for producing anti-HIV β-chemokines, wherein the increased production time provides for increased levels of β-chemokines to reduce the effects of HIV and/or reduce replication of HIV.

In another aspect, the invention relates to a method that increases production of chemokines, the method comprising:

administering to a subject a composition comprising at least one G1 phase arresting compound in an effective amount to delay entry of S-phase in the cell cycle of an activated T-cell, thereby increasing levels of chemokines in the G1 phase of cell cycle.

In still another aspect, the present invention relates to a method of combating a virus infection, comprising:

administering to a patient a composition comprising an effective amount of a G1 phase arresting compound to induce prolonged production of β-chemokines thereby increasing levels β-chemokines for antagonizing the function of a chemokine receptor.

In yet another aspect, the present invention relates to a method of maintaining durable viral control of HIV, the method comprising:

administering at least one antiviral agent and a G1 cell cycle arresting compound.

The antiviral agent may be an HIV entry inhibitors, such as TAK 799 or SCH-C both of which block viral binding to CCR5 receptors. Viral resistance to these CCR5 antagonist molecules has been shown to result from more efficient use of CCR5 by the virus (Trkola, et al., 2002). The fact that HIV-1 viruses that are resistant to CCR5 blockers are still dependant on CCR5 receptors for infection suggests that the increase in extracellular β-chemokines resulting from the use of G1 cell cycle agents will interfere with the growth and emergence of resistant viral variants, thereby increasing the antiviral durability of entry inhibitor therapy.

Another aspect of the present invention relates to a therapeutic method to reduce effects and replication of HIV in a HIV infected subject, the method comprising administering a G1 phase arresting agent alone and with at least one antiviral agent in a cyclic schedule or regime.

The cyclic schedule of the present invention may comprise:

a) administering a combination of at least one antiviral agent and at least one G1 phase arresting agent to the HIV infected subject for a first predetermined time period;

b) administering the at least one G1 phase arresting compound to the HIV infected subject for a second predetermined time period;

c) administering the combination of the antiviral agent and G1 phase arresting agent to the HIV infected subject for a predetermined third time period which is less than the first period;

d) administering the G1 phase arresting compound to the HIV infected subject for a fourth predetermined time period, which is less than the second time period; and

e) maintaining the cyclic schedule of steps c and d until an increase in components indicates rapid replication of the HIV virus. The components may include viral antigens, reduced T cells and any other indicator used by one skilled in the art to determine the progression of HIV.

The cyclic administering the antiviral agents and G1 phase arresting compounds may be maintained for an indefinite period of time with periodic evaluation of viral load.

In still a further aspect, the present invention relates to a method of preventing HIV in a subject potentially exposed to the HIV, the method comprising:

administering to the subject at least one G1 phase arresting compound in an effective amount to increase levels and/or availability of β-chemokines thereby inhibiting binding of HIV to β-chemokine receptors which, in turn, prevents HIV viral entry.

Another aspect of the present invention relates to a method to reduce dependency and/or effective amount of HIV antiviral agent by substituting the antiviral agent with a G1 phase arresting agent, augmenting the antiviral agent with a G1 phase arresting compound or substituting a portion of the antiviral agent with a G1 phase arresting compound. By substituting and/or augmenting antiviral agents with a G1 phase arresting compound, antiretroviral ARV therapy may be discontinued, amounts of antiviral agents can be reduced at least temporarily, and the ARV therapy is deintensified and simplified.

Other features and advantages of the invention will be apparent from the following detailed description, drawings and claims.

### BRIEF DESCRIPTION OF THE FIGURES

FIGS. 1A and B show kinetics of RANTES, MIP-1α, and MIP-1β secretion in activated PBMCs. (A) PBMCs (1×10⁶) were cultured in 1 ml of culture medium in the presence of PHA. Cultures were maintained for 72 h. Every 24 h, the entire culture medium was collected and replaced with fresh medium containing PHA. Supernatants were assayed for chemokine content by ELISA. DNA synthesis was measured by [³H]thymidine incorporation in PBMCs cultured in parallel under identical conditions. Results are the mean±SD of data obtained from three different donors. (B) PBMCs from four donors were cultured in the presence of PHA for 3 days and in the presence of IL-2 afterward. Culture supernatants were assayed for β-chemokine production by ELISA on days 3, 7, and 11. Values from each donor are the mean±SD of triplicate wells.

FIGS. 2A, B and C show HU treatment of PHA-activated PBMCs results in increased levels of secreted β-chemokines. PBMCs were cultured in the presence of PHA for 3 days and in the presence of IL-2 afterward. HU was added at the indicated concentrations at the beginning of the experiment and added fresh every time the medium was changed. Culture supernatants collected on days 3, 8, and 14 were assayed for chemokine production levels. Chemokine levels in the supernatants are expressed as ng/ml (A) and as ng per 10⁶ viable cells (B); cell number was monitored by trypan blue exclusion (C). Representative values of one of four experiments, each using PBMCs from a different donor, are shown. Values are means±SD of triplicate wells. *, P<0.01; #, P<0.05, compared with untreated control by Student's t test.

FIGS. 3A, B, C and D show treatment of activated PBMCs with G1 cytostatic drugs inducing G1 cell cycle arrest results in increased levels of extracellular β-chemokines. PHA-activated PBMCs were cultured in the presence of APH (A), SB (B), OL (C), or RC (D) at the indicated concentrations. Cultures were kept for 14 days, with medium changes every 3 or 4 days. Culture supernatants were tested for chemokine content by ELISA, and cell number was determined by trypan blue staining. Data show day 8 values for both APH and SB and day 3 values for RC and OL. Values are means±SD of triplicate wells. *, P<0.01; #, P<0.05, compared with untreated control by Student's t test.

FIGS. 4A, B, C, D and E show cell cycle arrest in G1, but not in G₂, results in increased extracellular levels of β-chemokines. Purified CD8 lymphocytes were activated by anti-CD3 and IL-2 treatment for 3 days. Activated cells were cultured in the presence of IL-2 medium containing HU at the indicated concentrations. After 24 and 48 h of the addition of HU, cell number was evaluated by trypan blue staining (A), newly synthesized DNA was measured by [³H]thymidine incorporation (B), percentage of cells in S phase was determined by propidium iodide staining (C), and β-chemokine levels were determined by ELISA (D). (E) Cell cycle arrest and chemokine production levels in the presence of nocodazole 48 h after addition of the drug. Results are single data values, representative of three independent experiments for HU and representative of two independent experiments in the case of nocodazole.

FIGS. 5A and B show supernatants collected from PBMCs exposed to HU contain suppressive factors that markedly inhibit HIV-1 BaL replication, whereas they only slightly affect the replication of HIV-1 IlIb. Activated lymphocytes from a seronegative donor were infected with HIV-1 BaL (A) or HIV-1 IIIb (B). Infected cells were cultured in IL-2 culture medium supplemented by 50% with supernatants collected from HU-treated PBMCs (CM/HU) or supernatants collected from untreated PBMCs (CM/control). In addition, a culture containing 100 μM HU in fresh medium was included. Virus replication was measured in the culture supernatant on day 7 after infection. Cell viability was assessed by the MTT assay. Data are means±SD of triplicate wells.

FIG. 6 shows that antiviral activity of supernatants collected from HU-exposed PBMCs is reversed by neutralizing antibodies against the β-chemokines RANTES, MIP-1α, and MIP-1β. Activated lymphocytes from a seronegative donor were infected with HIV-1 BaL. Infected cells were cultured in the presence of supernatants collected from PBMCs that had been cultured for 7 days in the presence of 100 μM HU (CM/HU). CM/HU was preincubated with a mixture of neutralizing antibodies (anti-RANTES, anti-MIP1α, and anti-MIP1β; indicated as nAb) or an IgG control before addition to the culture. Fresh medium containing CM/HU and the correspondent antibodies was added again on day 3 after infection. On day 7, viral replication was measured by a p24 assay, and cell viability was assessed by the MTT assay. Data are mean values±SD of duplicate wells.

FIG. 7 shows the effect of RAPA on proliferation of PBMCs. Purified PBMCs from normal donors were cultured in the presence of IL-2 and RAPA. On day 7, the extent of cell proliferation was measured by the MTT assay. Representative results obtained on one of two independent experiments, each using cells from four donors, are shown. For each donor, data values are mean±SD of three independent wells

FIGS. 8A and B show RAPA increases extracellular β-chemokine levels in PBMC cultures. (A) Donor PBMCs were cultured in the presence of IL-2 and RAPA for 10 days, at which time supernatants were evaluated for β-chemokine content by ELISA and then cells were also stained for CCR5 expression. Results shown in two donors are representative of four experiments using four different donors. *, P<0.01; #, P<0.05, compared with untreated control by Student's t test. (B) Effect of RAPA on extracellular levels of MIP-1β in cultures of CCR5-null PBMCs. Levels of MIP-1β protein in the presence and absence of RAPA were measured in supernatants of IL-2-stimulated PBMCs from a normal donor and from a donor homozygous for the Δ32 mutation in the CCR5 gene. Values were obtained on day 10 of culture and are means±SD of duplicate wells. Results are representative of two independent experiments, each using cells from two normal donors.

FIGS. 9A, B and C show that RAPA inhibits HIV-1 replication in PBMCs, and the antiviral activity in R5 HIV-1 is greater than in X4 HIV-1. (A) Seven-day RAPA-treated PBMCs were infected with HIV-1 IIIb or HIV-1 ADA. Infected cells were cultured in the presence of RAPA for 7 days, at which time virus replication was measured by p24 and cell viability was measured by the MTT assay. Results (means±SD of triplicate wells) are representative of seven independent experiments, each on cells from a different donor. (B) DNase-treated stocks of HIV-1 IIIb and HIV-1 ADA were used to infect PBMCs that had been treated with or without 100 nM RAPA. HIV-1 DNA sequences were amplified by PCR in cellular lysates prepared 24 h after infection. Amplified PCR products were detected with a radioactive probe. The symbol “+” indicates presence of RAPA in the PBMC culture before and after infection; “−” indicates no RAPA treatment. Amplification of β-actin sequences indicated same amount of cellular DNA among the different cell lysates (data not shown). NC denotes PCR negative control. (C) The antiviral activity of low concentrations of RAPA was investigated in a panel of R5 strains of HIV-1. Cell proliferation was assayed on uninfected cells from same donor cultured under identical conditions. Results (means±SD of triplicate wells) are representative of three independent experiments, each on different donor cells.

FIG. 10 shows that RAPA inhibits HIV-1 replication in MDMs. Purified monocytes were cultured for 5 days in the presence of RAPA. On day 5, cells were infected with HIV-1 ADA and cultured in the presence of RAPA for 14 additional days. On days 7, 10, and 14 after infection, virus growth was measured by the RT assay. On day 14, cell viability was determined by MTT. Results (means±SD) are representative of data obtained in three independent experiments, each using cells from a different donor.

FIG. 11 shows that RAPA enhances the antiviral activity of the CCR5 antagonist TAK-779. PBMCs that had been cultured in the absence or presence of RAPA (1, 10, and 100 nM) for 7 days were infected with HIV-1 ADA in the presence of 0.1 nM TAK-779. Infected cells were cultured in the presence of RAPA and 0.1 nM TAK-779. On day 7 after infection, virus production was measured by the p24 assay in the culture supernatant. Note the logarithmic scale in the y axis. Data represent means±SD of triplicate wells. Representative results obtained in one of three independent experiments are shown.

FIG. 12 shows that treatment of activated PBMCs with Vitamin E resulted in increased levels of β-chemokines.

FIG. 13 shows the effects of Vitamin E (alpha-tocopherol) on HIV-1 production upon activation of patient's resting CD4 T cells. Virus production (p24 antigen) was measured in the culture supernatants by ELISA on day 14. Log transformation of p24 values (pg/m/) were plotted. For statistical analysis, p24 negative samples were assigned a value of 6 pg/ml, which represents the detection limit of the p24 assay. Data values for each patient culture (with and without VE) are represented by symbols.

FIG. 14 shows the results of treatment with 2 mg/day of rapamycin and the increase of β-chemokines (RANTES) in five subjects with a return to baseline at day 28. (Results normalized for β-actin).

FIGS. 15A and B show that Rapamycin increases extracellular β-chemokine levels in cultured PBMCs. PBMCs from a healthy donor were cultured in the presence of IL-2 and Rapamycin. On day 7, chemokines content in the supernatant was measured by ELISA and cell viability was determined by the MTT assay.

### DETAILED DESCRIPTION OF PREFERRED EMBODIMENTS

A method of treating a viral infection is meant herein to include “prophylactic” treatment or “therapeutic” treatment. A “prophylactic” treatment is a treatment administered to a subject who does not exhibit signs of a disease or who exhibits early signs of the disease for the purpose of decreasing the risk of developing pathology associated with the disease. The term “therapeutic,” as used herein, means a treatment administered to a subject who exhibits signs of pathology for the purpose of diminishing or eliminating those signs.

The term “therapeutically effective amount,” as used herein means an amount of compound that is sufficient to provide a beneficial effect to the subject to which the compound is administered. A beneficial effect means rendering virus incompetent for replication, inhibition of viral replication, inhibition of infection of a further host cell, or increasing CD4 T-cell count, for example.

The term “a virally-targeted cell,” as used herein, means a cell in which virus is present and is infective or potentially infective and includes epithelial cells, nervous system cells, T-lymphocytes (activated or resting), macrophage, monocytes, tissue dendritic cells or the like.

The term “functional equivalent,” as used herein, means that the agent retains some or all of the biological activity of the corresponding compound.

The term “functional analog,” as used herein means compounds derived from a particular parent compound by straightforward substitutions that do not result in a substantial (i.e. more than 100×) loss in the biological activity of the parent compound, where such substitutions are modifications well-known to those skilled in the art, e.g., esterification, replacement of hydrogen by halogen, replacement of alkoxy by alkyl, replacement of alkyl by alkoxy, etc.

The Invention:

G1 Phase Arresting Compounds

The compositions of the present invention may include any G1 phase arresting agent that arrests, delays or prolongs cell-cycle activity in the G1 phase and/or G1-S interface of mononuclear cells. G1 phase arresting agents may include, but are not limited to, sodium butyrate, aphidicolin, hydroxyurea (HU), olomoucine, roscovitine, tocopherols, tocotrienols, rapamycin (RAPA) and/or functional analogs thereof.

The present invention employs a G1 phase arresting compound for administration to a subject suffering from a viral infection, wherein the compound prolongs the G1 phase of the cell cycle of an activated lymphocyte thereby providing an increase number of receptor-ligands to reduce replication of the viral infection.

Pharmaceutical Compositions Acceptable Derivatives and Salts

The present invention provides compositions comprising at least one G1 phase arresting compound and optionally at least one antiviral agent, as well as methods of preventing, treating and/or reducing the effects of HIV. The methods comprise administering said compositions comprising the G1 phase arresting compounds and optionally antiviral agents, wherein the two compounds can be administered, separately, simultaneously, concurrently or consecutively.

Pharmaceutically Acceptable Derivatives and Salts

The term “pharmaceutically acceptable derivative” is used herein to denote any pharmaceutically or pharmacologically acceptable salt, ester or salt of such ester of a compound according to the invention, or any compound which, upon administration to the recipient, is capable of providing (directly or indirectly) one ore more of the compounds according to the invention, or an antivirally active metabolite or residue thereof.

Preferred esters of the G1 phase arresting compounds of the invention include carboxylic acid esters in which the non-carbonyl moiety of the ester grouping is selected from straight or branched chain alkyl. e.g. n-propyl, t-butyl, n-butyl, alkoxyalkyl (e.g. methoxymethyl), aralkyl (e.g. benzyl), aryloxyalkyl (e.g. phenoxymethyl), aryl (e.g. phenyl optionally substituted by halogen, C₁₋₄alkyl or C₁₋₄ alkoxy or amino); sulfonate esters such as alkyl- or aralkylsulfonyl (e.g. methanesulfonyl); amino add esters (e.g. L-valyl or L-isoleucyl); and mono-, di- or triphosphate esters. The phosphate esters may be further esterified by, for example, a C₁₋₂₀ alcohol or reactive derivative thereof, or by a 2,3-di C₂₋₄ acyl glycerol.

Pharmaceutically acceptable salts include, without limitation, salts of organic carboxylic acids such as acetic, lactic, tartaric, malic, isethionic, lactobionic, p-aminobenzoic and succinic adds; organic sulfonic acids such as methanesulfonic, ethanesulfonic, benzenesulfonic and p-toluenesulfonic acids and inorganic adds such as hydrochloric, sulfuric, phosphoric and sulfarnic acids.

Anti-Viral Compounds

In one aspect the compositions and methods of the present invention further comprise a therapeutically effective amount of at least one antiviral agent, including, but not limited to nucleoside RT inhibitors, CCR5 inhibitors/antagonists, viral entry inhibitors and functional analogs thereof.

Preferably, the antiviral agent comprises nucleoside RT inhibitors, such as Zidovudine (ZDV, AZT), Lamivudine (3TC), Stavudine (d4T), Didanosine (ddl), Zalcitabine (ddC), Abacavir (ABC), Emirivine (FTC), Tenofovir (TDF), Delaviradine (DLV), Efavirenz (EFV), Nevirapine (NVP), Fuzeon (T-20), Saquinavir (SQV), Ritonavir (RTV), Indinavir (IDV), Nelfinavir (NFV), Amprenavir (APV), Lopinavir (LPV), Atazanavir, Combivir (ZDV/3TC), Kaletra (RTV/LPV), Trizivir (ZDV/3TC/ABC);

CCR5 inhibitors/antagonists, such as SCH-C, SCH-D, PRO 140, TAK 779, TAK-220, RANTES analogs, AK602, UK-427, 857, monoclonal antibodies;

viral entry inhibitors, such as Fuzeon (T-20), NB-2, NB-64, T-649, T-1249, SCH-C, SCH-D, PRO 140, TAK 779, TAK-220, RANTES analogs, AK602, UK-427, 857; and functional analogs thereof.

Antiviral Therapy

Although current treatment with antiretroviral (ARV) therapy causes suppression of HIV replication and results in improvements of immune function, it is limited by high costs, toxicities and is difficult to adhere to. Moreover, the chance of achieving long-term control of HIV infection with antiretroviral therapy alone seems very unlikely. To date, current antiretroviral therapy has been shown to be insufficient to completely eradicate HIV from infected individuals and there is no real data that the amount of residual virus is decreasing with time on typical antiretroviral therapy. Further, after stopping antiretroviral therapy, the viral load can rebound to higher levels than pretreatment viral loads (Davey, 1999; Dybul, et al., 2002 and 2001).

Antiretroviral therapy demands stringent adherence to complex dosing regimens. The rate of virological failure over a 6-month period of time has been demonstrated to be as high as 60% in patients that cannot achieve greater then 95% adherence. The combination of multiple adverse side effects associated with antiretroviral therapy and the availability of this treatment to only 1 in 20 of the estimated 33 million people infected world wide has prompted us to reconsider the current strategies for achieving the goals of HIV therapy.

Moreover, HIV therapy is now thought to be a life-long process. Therefore, it is crucial to develop effective treatments that can be successfully administered for long periods of time for the suppression of retroviruses, and in particular, the prevention and/or inhibition of HIV. Further, it would be desirable to eliminate, or at least minimize, the cytotoxicity associated with the administration of antiviral agents otherwise determined to be effective. It is generally recognized that the toxicity of an antiviral agent may be avoided or at least minimized by administration of a reduced dose of the antiviral agent; however, it is also recognized that the effectiveness of an antiviral agent generally decreases as the dose is reduced.

Thus, one embodiment of the present invention provides for reducing the dose of antiviral agents while maintaining or reducing viral load by using cyclic therapy and introducing the G1 cell cycle agents of the present invention to a dosing regime for an HIV infected subject. Specifically, the use of the G1 phase arresting compounds in combination with antiviral agents has shown promise to maintain viral suppression in a cycle therapy dosing program. By using 50% less medication, side effects associated with antiretroviral use have been shown to be reduced and adherence has shown to be increased. The other obvious impact is on overall cost of medications, which will facilitate expanding these drugs throughout the developed world.

By using our insight on the importance of G1 cell cycles in the treatment of HIV, manipulation of HIV cellular cycles can be used successfully to lengthen the off therapy periods of cyclic therapy. Further, HIV is decreased in the active and resting cell compartments. Resting lymphocytes are a major reservoir for HIV and thus it is important that antiretroviral therapy be capable of suppressing HIV in both resting and activated cells. Resting T cells can be infected by HIV at levels comparable to that of activated T cells. However, unlike the activated T cells, the viral DNA is only partially transcribed in resting T cells resulting in unintergrated proviral DNA. However, this proviral HIV DNA in the resting T cells may constitute a labile but inducible reservoir for activation. The importance of the activated cellular state for HIV replication coupled with the transient survival of replication competent unintergrated proviral intermediates raises the possibility of successful intervention aimed at both depleting the HIV DNA from the resting cell pool and also decreasing the state of cellular activation.

Although attempts using primarily protease inhibitor containing regimens have failed to reduce the overall burden of HIV, therapeutic interventions that are specifically aimed at preventing the persistence and renewal of the resting cell reservoir may be successful but have not yet been pursued. The failure to decrease the overall HIV burden on typical antiretroviral therapy may be attributed to the inadequate activity of antiviral agents and protease inhibitors in resting cells.

Thus, in one embodiment of the present invention, cyclic therapy is employed as an alternative approach designed to increase activity of antiviral agents, decrease drug cost and toxicity. Furthermore, since one component of the compositions of the present invention targets cellular machinery of the host, rather than the virus, the present inventors expect that viral resistance to this drug combination essentially would not occur.

A cycle antiviral therapy regime could run for about 12 weeks and then a G1 phase arresting compound is added or substituted for four weeks. If the viral load remains low or approximately constant, the cycles can be altered to reduce the time period of each cycle. The time period for consumption of the antiviral drugs can be reduced, if augmented with a G1 phase arresting compound. Furthermore, a time period can be introduced that includes no antiviral drugs and only a G1 phase arresting compound. This time period wherein no antiviral agents are consumed by the subject, provides the biological system of the subject sufficient time to repair or compensate for the toxic effects of the antiviral compound.

A proposed dosing program may include one week of consumption of antiviral agents plus a G1 phase arresting agent (3TC, Tenofovir (Tenofovir has improved potency in activated and resting T cells), and Sustiva, and G1 phase arresting compound HU), and then two weeks off of the antiviral agents but the G1 phase arresting agent is still consumed by the subject. Patients are monitored for immunological and virological parameters as well as the incidence of toxicity and side effects during both the treatment period and the interruption period. These cycles of 1 week on/2 weeks off of antiretroviral medications will continue for an appropriate treatment period with constant reevaluation of viral loads. Obviously, each subject will respond differently to such cycles and a physician knowing the dynamics of the HIV infection can determine the appropriate time period for each cycle.

Methods for Preventing and/or Treating a Viral Infection

The compositions and methods of the present invention can be used to prevent viral infection in a subject potentially exposed to the infection. The viral infections prevented by using the compositions and methods of the present invention are preferably retroviral infections, and are more preferably, HIV infections. G1 cell cycle agents for the prevention of HIV transmission either as single therapeutic agents or when used in combination with antiretroviral drugs and HIV vaccines may be used in the following settings:
1. Post blood borne exposure;
2. Post sexual exposure;
3. Mother to child transmission resulting from pregnancy, labor, delivery and through breast milk transmission; and
4. Augmentation of preventive HIV vaccine efficacy.

Further, the compositions and methods of the present invention can be used to treat HIV viral infections by reducing viral load and replication of the virus.

Still further the compositions and methods of the present invention can be used in combination with HIV vaccines to increase the efficacy of a vaccine in a subject. The methods comprise administering to a subject an immunizingly effective amount of one or more antigens against which an immune response is desired in the subject in conjunction with an amount of a G1 phase arresting agent effective to enhance the immune response against the antigen by increasing the levels of chemokines in the subject. In one aspect, the G1 phase arresting agent is administered to the subject concurrently with (e.g., in the same composition with) the antigen or antigens against which an immune response is desired.

In another, aspect, the G1 phase arresting agent is administered either before or after the administration of one or more antigens against which immunity is desired in the subject, but is administered within such time that the G1 phase arresting agent enhances the immune response to the one or more antigens. For example, the G1 phase arresting agent is suitably administered during the time that the subject mounts an immune response against the administered one or more antigens. The G1 phase arresting agent is preferably administered within 30 minutes, 1 hour, 5 hours, 10 hours, 1 day, and/or 2 days of (preferably, after) administration of the one or more antigens against which immunity is desired.

In yet another aspect, the G1 phase arresting agent is suitably administered for an extended period of time after the vaccine is administered as a chemo-prophylactic agent that maximizes the effectiveness and long-term protection of the vaccine.

The present invention further provides compositions comprising an immunizingly effective amount of one or more antigens and an amount of at least one G1 phase arresting agent effective to induce increased levels of chemokines.

Doses to be administered are variable according to the G1 phase arresting agent, the antiviral agent, the treatment period, frequency of administration, the host, and the nature and severity of the infection. The dose can be determined by one of skilled in the art without an undue amount of experimentation.

The compositions of the invention are administered in substantially non-toxic dosage concentrations sufficient to ensure the release of a sufficient dosage unit of the present combination into the patient to provide the desired inhibition of the HIV virus. The actual dosage administered will be determined by physical and physiological factors such as age, body weight, severity of condition, and/or clinical history of the patient. The active ingredients are ideally administered to achieve in vivo plasma concentrations of an antiviral agent of about 0.01 uM to about 100 uM, more preferably about 0.1 to 10 uM, and most preferably about 1-5 uM, and of a G1 phase arresting agent of about 1 uM-25 uM, more preferably about 2-20 uM, and most preferably about 5-10 uM.

For example, in the treatment of HIV-positive and AIDS patients, the methods of the present invention may use compositions to provide from about 0.005-500 mg/kg body weight/day of an antiviral agent, more preferably from about 0.1-200 mg/kg/day, and most preferably 1-50 mg/kg/day; and from about 0.01-1000 mg/kg body weight/day of a G1 phase arresting agent, more preferably from about 0.001-1000 mg/kg/day, or most preferably from about 0.5-50 mg/kg/day. Particular unit dosages of a G1 phase arresting agent and an antiviral agent of the present invention include 50 mg, 100 mg, 200 mg, 500 mg, and 1000 mg amounts, for example, formulated separately, or together as discussed infra.

It will be understood, however, that dosage levels that deviate from the ranges provided may also be suitable in the treatment of a given viral infection.

Therapeutic efficacy of the G1 phase arresting compounds can be determined by standard pharmaceutical procedures in cell cultures or experimental animals, e.g., for determining The LD₅₀ (The Dose Lethal To 50% Of The Population) and The ED₅₀ (the dose therapeutically effective in 50% of the population). The dose ratio between toxic and therapeutic effects is the therapeutic index and it can be expressed as the ratio LD₅₀/ED₅₀. Compounds, which exhibit large therapeutic indexes, are preferred. The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The desired dose is preferably presented as two, three, four, five, six or more sub-doses administered at appropriate intervals throughout the day. These sub-doses may be administered in unit dosage forms, for example, containing 0.01 to 1000 mg, preferably 1 mg to 50 mg, depending on the number of sub-doses, of the G1 phase arresting compound per unit dosage form.

While it is possible for the specific G1 phase arresting compound and antiviral agent to be administered individually, either sequentially or simultaneously, it is preferable to present them together, as combined in a pharmaceutical composition.

The compositions of the present invention may comprise both the above-discussed ingredients, together with one or more acceptable carriers thereof and optionally other therapeutic agents. Each carrier must be “pharmaceutically acceptable” in the sense of being compatible with the other ingredients of the formulation and not injurious to the subject.

The present invention provides a method for the treatment or prophylaxis of a viral infection such as retroviral infections which may be treated or prevented in accordance with the invention include human retroviral infections such as human immunodeficiency virus (HIV), HIV-1, and HIV-2. The specific G1 phase arresting compounds, compositions and methods according to the invention are especially useful for the treatment of AIDS and related HIV-positive conditions. The compounds of the present invention are also useful for the treatment of asymptomatic infections or diseases in humans caused by or associated with human retroviruses.

The therapeutic compositions according to the present invention may be employed in combination with other-therapeutic agents for the treatment of viral infections or conditions. Examples of such further therapeutic agents include agents that are effective for the treatment of viral infections or associated conditions such as immunomodulatory agents such as thymosin, ribonucleotide reductase inhibitors such as 2-acetylpyridine 5-[(2-chloroanilino)thiocarbonyl)thiocarbonohydrazone, interferons such as alpha-interferon, 1-beta-D-arabinofuranosyl-5-(1-propynyl)uracil, 3′-azido-3′-deoxythymidine, ribavirin and phosphonoformic acid.

Routes of Administration

The compositions according to the present invention, may be administered for therapy by any suitable route including oral, rectal, nasal, topical (including transdermal, buccal and sublingual), vaginal and parentertal (including subcutaneous, intramuscular, intravenous and intradermal). It will be appreciated that the preferred route will vary with the condition and age of the recipient, the nature of the infection and the chosen active ingredient.

Pharmaceutical formulations of the present invention include those suitable for oral, rectal, nasal, topical (including transdermal, buccal and sublingual), vaginal or parenteral (including subcutaneous, intramuscular, intravenous and intradermal) administration.

The formulations may conveniently be presented in unit dosage form and may be prepared by methods known in the art of pharmacy. Such methods include the step of bringing into association the G1 phase arresting compound and optionally an antiviral agent with the carrier. The carrier optionally comprises one or more accessory ingredients. In general, the formulations are prepared by uniformly and intimately bringing into association the separate ingredients with liquid carriers or finely divided solid carriers or both, and then if necessary shaping the product.

Compositions suitable for transdernal administration may be presented as discrete patches adapted to remain in intimate contact with the epidermis of the recipient for a prolonged period of time. Such patches suitably contain the G1 phase arresting compound and optionally an antiviral agent: 1) in an optionally buffered, aqueous solution; or 2) dissolved and/or dispersed in an adhesive; or 3) dispersed in a polymer. A suitable concentration of each synergistic ingredient is about 1% to 25%, preferably about 5 to 15%.

Formulations of the present invention suitable for oral administration may be presented as discrete units such as capsules, caches or tablets, each containing a predetermined amount of the ingredients; as a powder or granules; as a solution or a suspension in an aqueous or non-aqueous liquid; or as an oil-in-water liquid emulsion or a water-in-oil liquid emulsion.

A tablet may be made by compression or molding, optionally with one or more accessory ingredients. Compressed tablets may be prepared by compressing in a suitable machine the G1 phase arresting compound and antiviral agent in a free-flowing form such as a powder or granules, optionally mixed with a binder (e.g. povidone, gelatin, hydroxypropylmethyl cellulose), lubricant, inert diluent, preservatives, disintegrant (e.g. sodium starch glycollate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of one or more of the synergistic ingredients therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising one or more of the G1 phase arresting compounds and optionally an antiviral agent in a flavored basis, usually sucrose or acacia; pastilles comprising one or more of the ingredients in an inert basis such as gelatin and glycerin, or sucrose and acacia; and mouthwashes comprising the one or more of the ingredients in a suitable liquid carrier.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing, in addition to the one or more of the compounds of the present invention, such carriers as are known in the art to be appropriate.

Formulations suitable for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multidose sealed containers, for example, ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

For a perinatal subject, the drug combination of the present invention may be, for example, administered orally after 36 weeks of pregnancy and continued through delivery. Interventions around the time of late gestation and delivery (when the majority of transmissions are thought to occur) are most efficacious.

In addition to the compositions described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres that offer the possibility of local noninvasive delivery of drugs over an extended period of time. The administered therapeutic is slowly released from these microspheres and taken up by surrounding tissue cells (e.g. endothelial cells).

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration.

Suitable G1 cell cycle agents, can be used in HIV treatment strategies that allow for continued viral suppression to be maintained with less dependence on combination antiretroviral (ARV) therapy. The current goal of ARV is to obtain viral suppression as low as possible for as long as possible. Requiring less frequent dosing or a decreased quantity of ARV to control viral suppression directly addresses the problems, set forth below, associated with achieving the current goals of antiretroviral therapy including:
1. Current regimens of HAART are cumbersome and complicated and require sustained tolerance and strict adherence to 3 or more drugs.
2. Long term tight adherence may be impossible for most patients.
3. Long term tolerance to accumulating medication toxicities may be impossible for most patients.
4. Current treatment guidelines for HIV infection recommend a relatively late initiation of HAART because of the inability to eradicate the infection with HARRT alone and the risk of drug-related side-effects, including serious metabolic syndromes.
5. Some patients who have not been treated until later stages of the disease will have a high level of viral load, which could increase the risk of transmission and cause a public health problem.
6. Lastly, the vast majority of HIV infected people worldwide have no access to antiretroviral drugs due mostly to cost.

By incorporating G1 cell cycle agents into therapeutic approaches with the focus shifted towards maintaining long term viral control, with less complex, less toxic, and more affordable regimens, that can be applicable throughout the world. The present invention that targets the G1 cellular cycle to increase extracellular levels of chemokines can be used safely and successfully to maintain viral suppression in chronic HIV-1 infection without the need of continuous therapy with multiple antiretroviral drugs. These results have a positive impact on cost, side effects, and availability of HIV therapy.

The present invention is further illustrated by the following examples that should not be construed as limiting in any way.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization(B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

Methods and Materials

Tissue Culture.

PBMCs were separated from whole blood of HIV-1 seronegative donors by density centrifugation with Ficoll Histopaque (Sigma). Cells were cultured in complete medium consisting of RPMI medium 1640 supplemented with 10% heat-inactivated FBS, 2 mM glutamine, and penicillin/streptomycin (Invitrogen). In some experiments, purified CD8⁺ lymphocytes obtained by negative selection using the Human CD8⁺ T Cell Enrichment Mixture (StemCell Technologies, Vancouver) were used. Cell purity measured by flow cytometry was >80% among different donor purifications.

Cells were activated by culture for 72 h under three different conditions: phytohemagglutinin (PHA, 2.5 μg/ml; Roche, Gipf-Oberfrick, Switzerland), anti-CD3 antibody (1 μg/ml; Coulter) plus 100 units/ml recombinant IL-2 (Roche), or staphylococcal enterotoxin B at 0.03 μg/ml (Sigma). Activated cells were cultured in complete medium supplemented with recombinant IL-2 (100 units/ml), and medium was changed every 3 or 4 days.

Cell proliferation was measured by the trypan blue staining viability test, [³H]thymidine incorporation in DNA, and the 3-(4,5-dimethylthiazol-2-yl)-2,5-diphenyl tetrazolium bromide (MTT) assay (Roche).

Measurement of β-Chemokine Levels and Assessment of Cell Cycle Arrest.

The impact of cell cycle arrest in β-chemokine levels was evaluated by measuring chemokine levels in supernatants of cell cultures containing compounds known to cause cell cycle arrest. Levels of the β-chemokines RANTES, MIP-1α, and MIP-1β were measured by using commercial ELISA kits (R &D Systems). Cell cycle arrest in G1 was induced by culturing of the cells in the presence of aphidicolin (APH), sodium butyrate (SB), hydroxyurea (HU), roscovitine (RC), or olomoucine (OL). Arrest in late S phase was induced by culture of cells in the presence of resveratrol. G₂ cycle arrest was induced by the compounds nocodazole and Colcemid. All compounds were purchased from Sigma except RC and OL, which were from Calbiochem. Arrest of cell cycle progression in the presence of G1 cytostatic agents was measured by propidium iodide staining followed by fluorescence-activated cell sorter (FACS) analysis (Noguchi, et al., 1991). This method may be used to test any compound for ability to arrest the G1 phase in a cell cycle.

Assessment of HIV-1-Suppressive Activity in Supernatants Collected from HU-Treated PBMCs.

The antiviral activity of the supernatants collected from cultures of PBMCs that had been exposed to 100 μM HU for 7 days [supernatants referred to as conditioned medium (CM)] was evaluated in PBMCs infected with HIV-1 BaL and HIV-1 IIIb. Briefly, PHA-activated PBMCs were infected with each virus at 100 tissue culture 50% infective dose units (TCID₅₀)/10⁶ PBMCs or 10 TCID₅₀/10⁶ PBMCs for 2 h at 37° C. Infected cells were cultured in IL-2 medium alone, IL-2 medium with 100 μM HU, IL-2 medium containing 50% supernatant from HU-treated PBMCs (CM/HU), or IL-2 medium containing 50% supernatant from control-treated PBMCs (CM/control). On day 3 after infection, culture medium was replaced with fresh medium of the same kind as on day 1. Viral growth (measured by p24 levels in the supernatant) and cell viability (assayed by MTT) were determined on day 7 after infection. To determine the role of the β-chemokines RANTES, MIP-1α, and MIP-1β in the antiviral activity found in supernatants of HU-exposed PBMCs, the antiviral activities of such supernatants were evaluated in the presence of neutralizing antibodies against all three chemokines as described (Kay, et al., 1983).

### Example 1

Kinetics of β-Chemokine Secretion on Activated PBMCs.

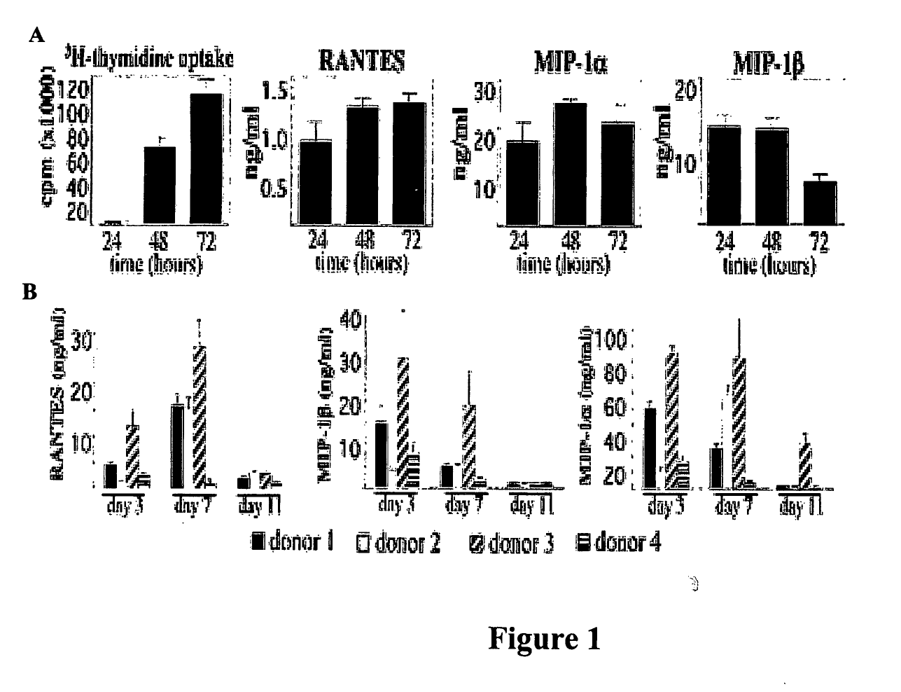

To determine the kinetics of β-chemokine secretion in relation to DNA synthesis on cellular activation, PBMCs were activated by PHA treatment for 72 h. At 24 h after plating the cells, the entire supernatant was collected, and fresh medium containing PHA was added to the culture. Twenty-four hours later (48 h from the time cells were plated), supernatants again were collected and PHA medium was added. Supernatants were collected for the last time after 72 h. Thus, this experiment measured the amount of chemckine released to the culture medium during each 24-h interval, not the continuous accumulation of chemokine over 48 or 72 h of exposure to PHA. Cellular DNA synthesis was measured by assaying [³H]thymidine incorporation in parallel wells at 24, 48, and 72 h after plating of the cells. Results are shown in FIG. 1A. At 24 h after the beginning of PHA treatment, chemokine protein concentration in the culture supernatants was 850, 17,200, and 13,300 pg/ml for RANTES, MIP-1α, and MIP-1β, respectively (average values from three different donors). Slightly increased values in RANTES and MIP-1α secretion were detected during the 24- to 48-h culture period, whereas the MIP-1β values remained constant. During the 48- to 72-h period, levels of RANTES and MIP-1α secretion were unchanged, whereas the secretion of MIP-1β decreased. Synthesis of cellular DNA was almost undetectable at 24 h (645 cpm) and increased considerably by 48 and 72 h (62,474 and 106,402 cpm, respectively). These data show that considerable protein amounts of MIP-1α, MIP-1β, and, to a lower extent, RANTES are present in the culture supernatant of PBMCs at 24 h after activation, a time at which cellular DNA synthesis is minimal.

The profile of β-chemokine secretion next was evaluated in cultures of activated PBMCs maintained in the presence of IL-2 for several days. PBMCs were activated with PHA for 3 days and then cultured in the presence of IL-2 for 8 additional days. At days 3, 7, and 11, chemokine content in the culture fluid was measured (FIG. 1B). Although variability was observed among different donors, RANTES levels usually reached a peak on day 7 after activation. In contrast, MIP-1α and MIP-1β levels peaked on day 3 or day 7, depending on the donor. Levels of all three chemokines were low by day 11. Taken together, these data indicate that secretion of the β-chemokines by PBMCs in response to activation starts before lymphocytes enter the DNA synthesis phase of the cell cycle (S phase), reaches a peak by day 3 or 7, and then declines to low levels.

### Example 2

Treatment of PBMC Cultures with Compounds That Arrest the Cell Cycle in G1 Results in Increased Levels of Secreted β-Chemokines.

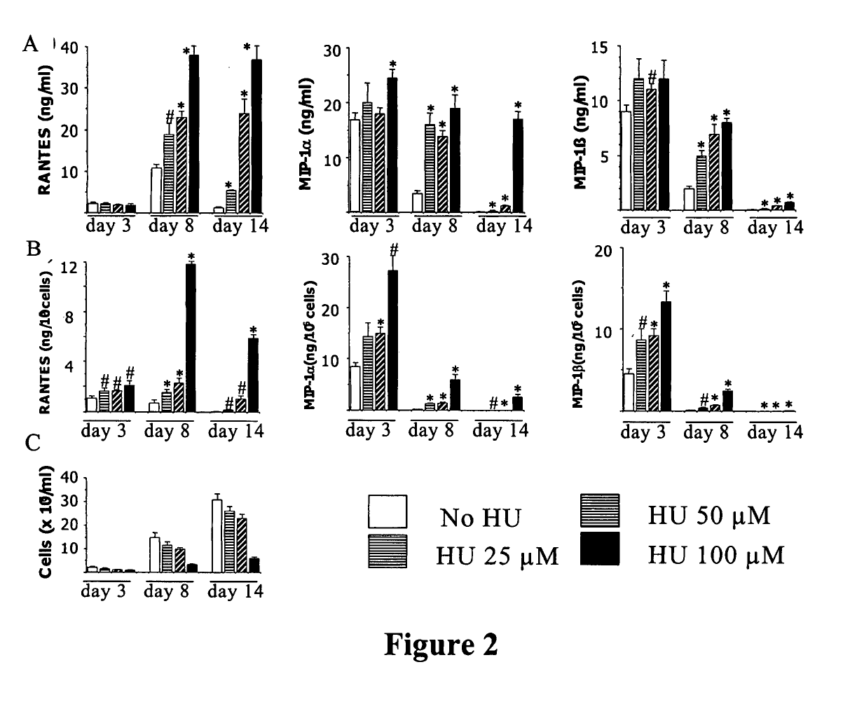

Because the previous experiments indicated that β-chemokine secretion by activated PBMCs begins before DNA synthesis occurs, it was further tested to determine whether delay of entry in the S phase of the cell cycle results in an overall increase in chemokine levels. To this end, β-chemokine levels (production or availability) by activated PBMCs cultured in the presence of HU was investigated. HU is a G1 cytostatic drug that, by depleting intracellular nucleotide pools, arrests cell cycle progression in late G1 (Lori, et al., 1994). FIG. 2 shows chemokine levels by activated PBMCs cultured in the presence of different concentrations of HU for 14 days. HU treatment resulted in increased concentrations (ng/ml) of RANTES, MIP-1α, and MIP-1β in the culture supernatants in a dose-dependent manner (FIG. 2A). In the representative experiment depicted in FIG. 2, day 8 chemokine levels in cultures containing 100 μM HU were increased 3.4-fold for RANTES, 5.4-fold for MIP-1α, and 4-fold for MIP-1β compared with the untreated control. Because HU inhibits lymphocyte proliferation, chemokine values also were expressed as chemokine amount per viable cell. As expected, cell numbers were lower in the presence of the drug (FIG. 2C). Chemokine levels expressed as ng per 10⁶ cells indicated increases of 16.2-, 25.4-, and 18.4-fold for RANTES, MIP-1α, and MIP-1β, respectively, in the presence of 100 μM HU (FIG. 2B). Similar increases were observed in PBMC cultures from the other three donors, and the increases were evident when chemokine values were expressed either as ng/ml or as ng per 10⁶ viable cells (data not shown).

Similarly, HU treatment increased chemokine levels in PBMCs that had been activated by cross linking of the T cell antigen receptor/CD3 complex with anti-CD3 antibodies or by occupancy of the T cell antigen receptor with the super antigen staphylococcal enterotoxin B (data not shown).

### Example 3

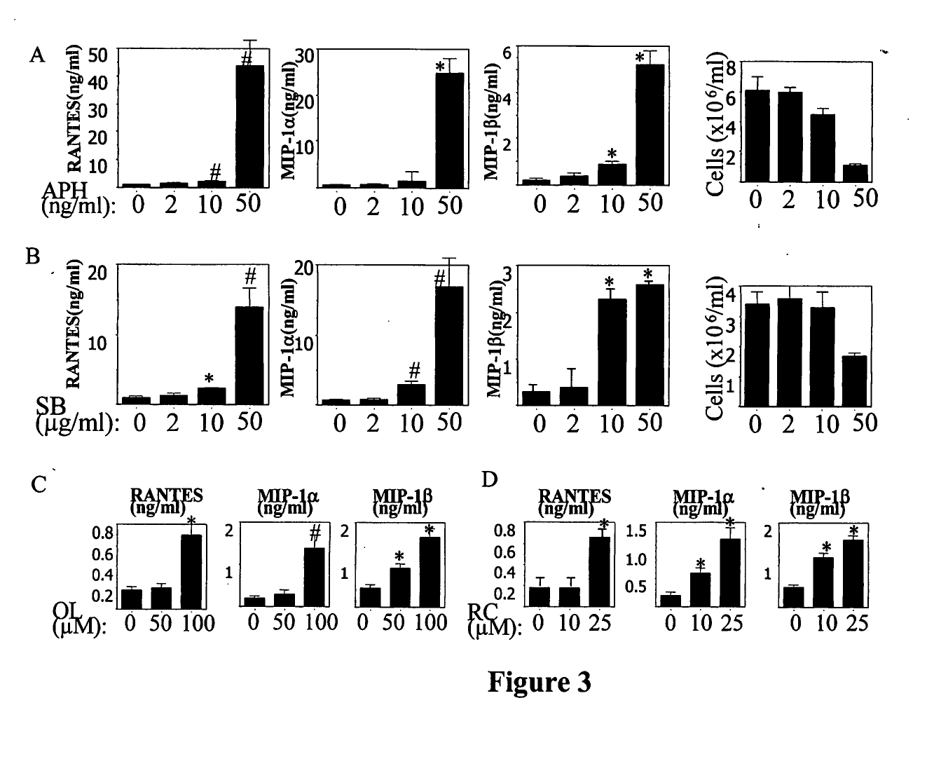

Having demonstrated that HU treatment of activated PBMCs results in increased chemokine levels, observations were extended to other G1 cytostatic agents that, as does HU, arrest cell cycle progression before DNA synthesis occurs. The agents evaluated were SB, APH, RC, and OL. SB and APH arrest the cell cycle in early and late G1, respectively (Korin, et al., 1998; Koostra, et al., 2000). RC and OL are purine-derivative drugs that arrest cell cycle progression in late G1 through inhibition of cyclin-dependent kinases (CDKs) (Gray, et al., 1999). FIG. 3A shows chemokine levels produced by activated PBMCs cultured in the presence of APH. The number of viable cells and chemokine levels are depicted. Increased chemokine values were observed at 0.5 μM APH, a drug concentration that exhibited G1 cytostatic effects as manifested by reduced cell proliferation. FIG. 3B shows chemokine levels produced by activated PBMCs cultured in the presence of SB. As was the case with APH, increased chemokine levels were detected at drug concentrations of SB exerting G1 cytostatic activity. Similarly, exposure of activated PBMCs to the CDK inhibitors RC and OL resulted in increased chemokine levels at G1 cytostatic concentrations of the drugs (FIG. 3C and data not shown). These experiments indicate that treatment of activated PBMCs with compounds that arrest the cell cycle in the G1 phase results in increased levels of extracellular β-chemokines.

### Example 4

Up-Regulation of β-Chemokine Levels in Supernatants of CD8 Lymphocyte Cultures Is Specific to Cell Cycle Arrest in G1.

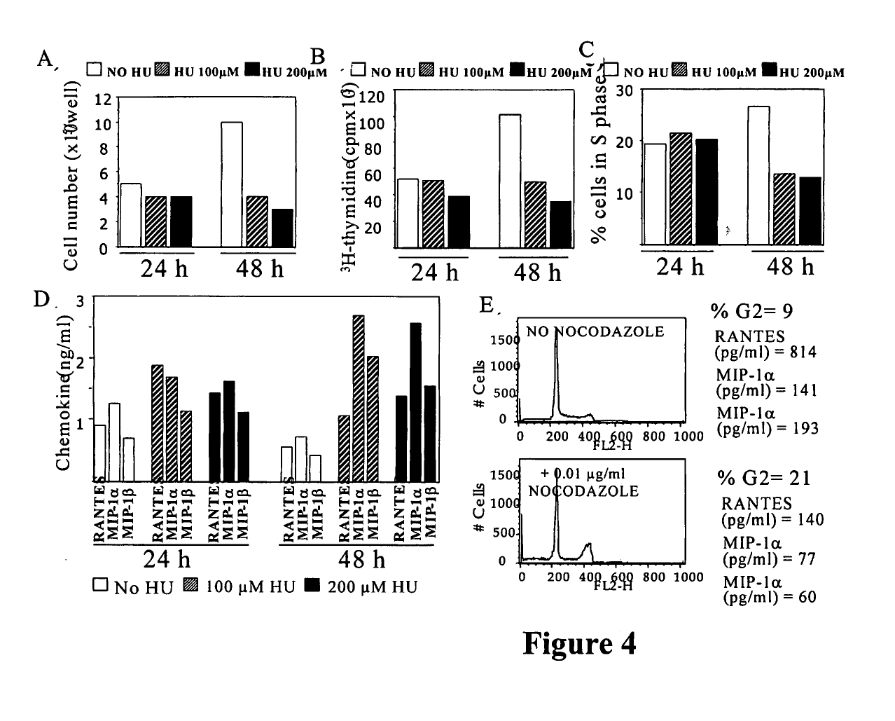

In the experiments described thus far, total PBMCs had been used. To demonstrate that arrest of the cell cycle in CD8 lymphocytes (the main cell type producer of the anti-HIV chemokines) results in increased chemokine levels, chemokine production by purified CD8 lymphocytes exposed to HU was evaluated next. Negatively selected CD8⁺ lymphocytes were activated by anti-CD3 plus IL-2 treatment for 3 days. Activated cells were cultured in the presence of IL-2 and HU (100 and 200 μM) for 24 or 48 h, time points at which cell proliferation and supernatant chemokine levels were assayed. Cell proliferation was determined by trypan blue staining, [³H]thymidine incorporation in DNA, and percentage of cells in S phase as assessed by propidium iodide staining (FIG. 4). HU cytostatic effects were evident after 48 h of exposure to the drug because both cell number and thymidine incorporation doubled between 24 and 48 h in the absence of HU, whereas they remained constant or decreased in the presence of the drug. Similarly, the percentage of cells in S phase increased in the absence of HU, whereas it decreased in its presence. CD8⁺ lymphocyte cycle arrest by HU resulted in increased levels of RANTES, MIP-1α, and MIP-1β after 24 and 48 h of exposure to the drug. At 48 h, levels of RANTES, MIP-1α, and MIP-1β increased by 1.9-, 3.7-, and 4.7-fold, respectively, in the presence of 100 μM HU. Slightly lower increases in chemokine production levels were found in CD8 cells exposed to 200 μM HU for 48 h, a drug concentration that resulted in a slightly lower number of viable cells shown by trypan blue staining in two of three donors examined (FIG. 4 and data not shown). In summary, these data demonstrate that arrest of cell cycle progression in CD8⁺ lymphocytes by HU treatment results in increased levels of RANTES, MIP-1α, and MIP-1β.

To investigate whether increased levels of supernatant chemokines upon cell cycle arrest are cell cycle phase-specific, the effect of G₂ arrest in chemokine production next was evaluated. Activated CD8 lymphocytes were arrested in G₂ by treatment with 0.01 μg/ml nocodazole (Gualberto, et al., 1998). As shown in FIG. 4E, treatment of activated CD8 cells with nocodazole for 48 h resulted in accumulation of cells in G₂ compared with the untreated control. However, chemokine content in treated cultures was lower than in the untreated controls. The same results were obtained on induction of G₂ cycle arrest by Colcemid (data not shown). Similarly, cell cycle arrest in late S by 10 μM resveratrol resulted in chemokine levels that were lower than the untreated control (data not shown). Together, these data suggest that increased chemokine levels are specific to cell cycle arrest in G1.

### Example 5

Supernatants Collected from PBMCs Arrested in G1 Inhibit HIV-1 BaL Replication.

To determine whether the augmented chemokine levels found upon G1 cell cycle arrest possess any antiviral activity, supernatants were harvested from cultures of activated PBMCs that had been exposed to 100 μM HU for 7 days. These culture supernatants were referred to as CM/HU. Culture supernatants from activated PBMCs cultured under the same conditions in the absence of HU (CM/control) also were harvested. Supernatants were harvested on day 7 because previous experiments (FIG. 2) indicated that high levels of RANTES, MIP-1α, and MIP-1β are present at this time point.

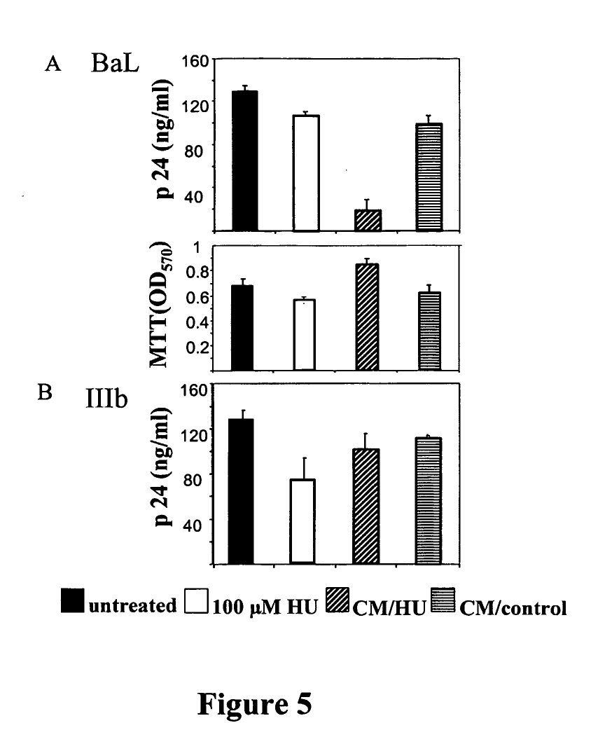

The antiviral activity of the CM was evaluated in the infection of PBMCs with HIV-1 BaL (a prototype R5-using virus) and HIV-1 IIIb (a prototype X4-using virus). Activated PBMCs from two seronegative donors were infected with each virus, and the infected cells were cultured in medium supplemented by 50% with CM/HU or CM/control. An additional culture was set up by culturing infected cells in fresh medium supplemented with 100 μM HU, the same concentration of HU as the one present in the CM/HU medium. Virus replication and cell viability were measured on day 7 after infection. Results obtained in one of the donors are shown in FIGS. 5A and B. Culture of infected PBMCs in the presence of CM/HU fluid suppressed HIV-1 BaL replication by 85% compared with the untreated control, whereas the CM/control fluid reduced virus replication by 24%. The control containing HU at the same concentration as the CM/HU inhibited HIV-1 BaL replication by 8%. Cell viability (MTT assay) was not affected by the addition of CM to the cultures. In contrast to the results obtained with HIV-1 BaL, the CM/HU and CM/control fluids suppressed HIV-1 IIIb by 22% and 10%, respectively. These data indicate that activated PBMCs grown in the presence of HU for several days release factors that strongly inhibit HIV-1 BaL replication, whereas they have a much lesser effect on the replication of HIV-1 IIIb. In addition, these results suggest that factors present in the supernatants, but not HU per se, are responsible for the inhibition of HIV-1 BaL.

### Example 6

The Antiviral Activity Present in Supernatants of Lymphocytes Arrested in G1 Phase by HU Treatment Is Due to RANTES, MIP-1α, and MIP-1β.

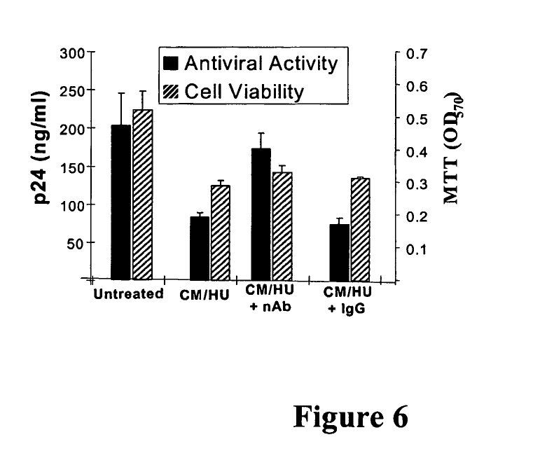

The above results demonstrated selective inhibition of the R5-using virus HIV-1 BaL by supernatants collected from HU-treated PBMCs. In addition, these results suggested that the β-chemokines RANTES, MIP-1α, and MIP-1β were the likely suppressive factors accounting for viral inhibition. To confirm that an increase in β-chemokine levels in the CM was responsible for the observed inhibition of HIV-1 BaL replication, the antiviral activity of the CM in the presence of a mixture of neutralizing antibodies to all three chemokines was assessed. As can be seen in the experiment depicted in FIG. 6, CM/HU fluid inhibited HIV-1 BaL replication by ≈60%. However, the antiviral activity of CM/HU was only ≈10% in the presence of the neutralizing antibodies mixture. An IgG antibody control did not affect the antiviral activity of the CM/HU fluid. In a different experiment, using cells from a different donor, the neutralizing antibodies mixture similarly abrogated the antiviral activity of the CM/HU supernatant (data not shown). These data demonstrate that the antiviral activity found in culture supernatants of HU-treated PBMCs primarily is due to the presence of the β-chemokines RANTES, MIP-1α, and MIP-1β.

The above-described experiments confirmed the presence of high levels of β-chemokines in supernatants of PBMC cultures 24 h after activation, a time at which cellular DNA synthesis could not be detected (FIG. 1A). By arresting cell cycle progression of PHA-activated PBMCs in G1 phase by treatment with G1 cytostatic drugs, increased levels of RANTES, MIP-1α, and MIP-1β were found in the culture supernatants. Increased levels of β-chemokines were specific to compounds that caused cell cycle arrest in G1, and the arrest of the cell cycle in late S (by resveratrol) or in G₂ (by nocodazole or Colcemid) did not result in such increases. The G1-arresting agents that in the present study demonstrated up-regulation of β-chemokine levels were SB, HU, RC, and OL.

HU then was selected for additional experiments as a representative agent. Simultaneous analyses of chemokine production levels and induction of G1 arrest by HU in purified CD8⁺ lymphocytes (monitored by cell number, thymidine incorporation, and percentage of cells in S phase) confirmed the results obtained in total PBMCs.

Supernatants collected from cultures of PBMCs (uninfected subjects) that had been exposed to HU for several days, referred to as CM in our experiments, were able to markedly suppress the replication of HIV-1 BaL in PBMCs. A mixture of neutralizing antibodies against all three chemokines abrogated the anti-HIV-1 BaL activity of the CM, thus demonstrating that the β-chemokines were responsible for the antiviral effect. When HU was added to fresh medium at the same concentration as in the CM, an only minor antiviral effect (<10%) was found in the replication of HIV-1 BaL. These data suggested that the antiviral effect observed was not due to inhibition of the virus reverse transcription step by HU-mediated depletion of nucleotide pools.

Taking our results together, we have described an approach to increase the concentration of the CCR5 ligands RANTES, MIP-1α, and MIP-1β in cultures of PBMCs or CD8⁺ lymphocytes. This approach involves the transient arrest of activated cells in the G1 phase of the cell cycle by using low concentrations of G1 cytostatic agents.

In summary, induction of anti-HIV chemokines by G1 cytostatic drugs that, by themselves, potentiate the antiviral activities of anti-HIV drugs offers an additional strategy with which to control replication of HIV-1. This may be a particularly attractive strategy with which to inhibit the virus in African countries in which subtype C is prevalent, because these viruses use CCR5 and not CXCR4 receptors (Ping, et al., 1999).

Further experiments were conducted to show that Rapamycin, also a G1 phase arresting agent causes accumulation of anti-HIV β-chemokines.

Methods and Materials

Cell Culture and Flow Cytometry.

Cultures of peripheral blood mononuclear cells (PBMCs) and monocyte-derived macrophages (MDMs) were performed on normal donors as described (Poli, et al. 1993, Perno, et al., 1993). PBMCs were maintained in the presence of 100 units/ml rhIL-2 (Roche Molecular Biochemicals). Cell viability was determined by Trypan blue staining or by the MTT assay (Roche Molecular Biochemicals). RAPA was purchased from Calbiochem. The CCR5 antagonist TAK-779 was obtained from the National Institutes of Health AIDS Research and Reference Reagent Program (Rockville, Md.). CCR5 surface expression was measured on PBMCs cultured in the presence of IL-2 for 7-10 days. Staining was done as described (Lane, et al. 1999), but using CCR5 mAb 182 (R & D Systems). Background staining was determined by adding an isotype-matched control (IgG2b, R&D Systems) instead of the anti-CCR5 mAb. Data were acquired by using a FACS Calibur flow cytometer (BD Biosciences) and analyzed by using FLOWJO (Tree Star, San Carlos, Calif.).

Levels of the β-chemokines MIP-1α, MIP-1β and RANTES were measured in culture supernatants by using ELISA kits (R & D Systems).

Infectivity Assays.

The following viruses were used in infection experiments, including HIV-1 IIIb, HIV-1 ADA, HIV-1 BaL, HIV-1 JRFL, HIV-1 JRCSF, and HIV-1 SF162. HIV-1 IIIb is a T cell line-adapted lab strain that uses CXCR4 for entry into cells, whereas the rest are isolates that use CCR5. Viruses were obtained from the National Institutes of Health AIDS Research and Reference Reagent Program.

For infection of PBMCs, fresh donor PBMCs were cultured for 7 days in medium containing IL-2. On day 7, cells were exposed to the virus for 3 h. Nonadsorbed virus was removed by washing cells with PBS three times. Infected cells were cultured in IL-2 medium. Infection of MDMs was carried out as described before (Pemo, et al. 1993). Unless otherwise indicated, PBMCs were infected by using a moi of 0.001, and monocyte-derived macrophages (MDMs) were infected by using a moi of 0.002. Virus growth was monitored in culture supernatants by measuring p24 antigen levels by ELISA (NEN) or by measuring viral RT activity in an RT assay (Willey, et al., 1988).

PCR Methodologies.

Amplification of CCR5 and β-chemokine RNA sequences was performed by RT-PCR by known methods. In some experiments, the effect of RAPA treatment on virus entry in PBMCs was investigated by DNA PCR. Briefly, PBMCs that had been treated with IL-2 and RAPA for 7 days were infected for 3 h with HIV-1 IIIb or HIV-1 ADA at an moi of 0.05. Virus inocula had been first filtered through a 0.22-μm filter and then treated with DNase (10 μg/ml) for 30 min at 37° C. to decontaminate the inoculum of HIV-1 DNA. Infected cells were washed extensively to remove residual virus. At 24 h after infection, cell lysates were prepared, and aliquots were amplified by DNA PCR using primer pair M661/M667 (Zack, et al. 1990). Amplified products were detected by liquid hybridization using a ³²P-labeled probe (Spina, et al., 1995). Intensities of hybridization signals were measured in a phosphoimager. β-Actin primers were used to control for DNA amount input in the sample.

### Example 7

Effect of RAPA on PBMC Proliferation and Viability.

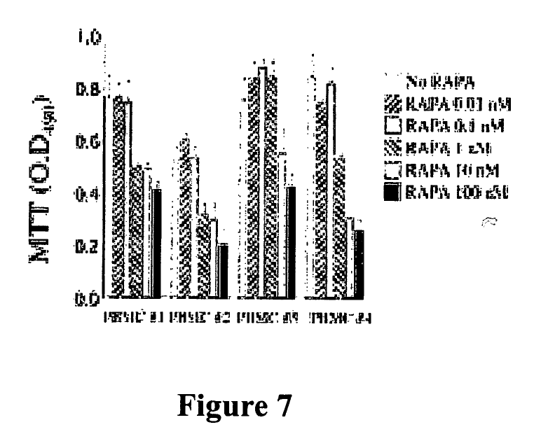

Donor PBMCs were cultured in the presence of IL-2 and RAPA (10-fold serial dilutions, from 10⁴ to 0.01 nM). Reduced proliferation, measured by the MTT assay on day 7, was detected at drug concentrations ≧1 nM (FIG. 7). Drug toxicity was observed at drug concentrations above 1 nM (data not shown).

### Example 8

RAPA Increases Extracellular Levels of MIP-1α and MIP-1β in PBMC Cultures.

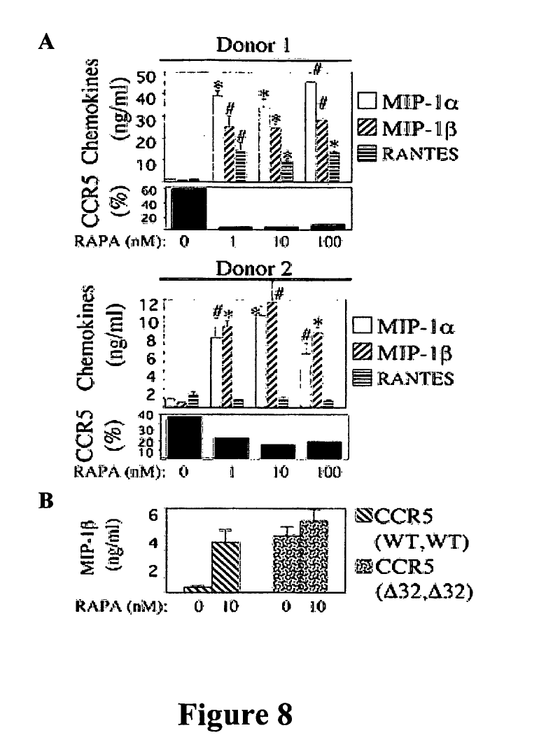

We next measured levels of the CCR5 ligands MIP-1α, MIP-1β, and RANTES in supernatants of RAPA-treated PBMC cultures. PBMCs from four donors were cultured in the presence of IL-2 and RAPA for 10 days. When chemokine content in culture supernatants was measured, it was found that MIP-1α and MIP-1β levels were higher in the presence of RAPA than in its absence in all four donors. Among the different donors, RAPA-treated cultures contained 6-39-fold higher levels of MIP-1α than untreated cultures. Similarly, MIP-1β levels were increased 17-47-fold in the presence of RAPA as compared with untreated controls. In contrast, levels of RANTES in the presence of RAPA were increased in two donors while remaining unchanged or even decreasing in the others. Chemokine results obtained in two of the donors, showing disparity of RANTES levels in the presence of RAPA, are depicted in FIG. 8A.

The effect of RAPA on MIP-1β protein levels provided information regarding the mechanism by which RAPA increases β-chemokine levels. To this end, stimulated PBMCs from two normal donors and from a CCR5-null donor were cultured in the presence of RAPA as in previous experiments. MIP-1β levels in supernatants were evaluated on day 10. Representative results obtained in one of the normal donors are shown next to the results obtained on the CCR5-null donor (FIG. 8B). In the normal donor, RAPA treatment resulted in an increased level of MIP-1β protein (9.3-fold increase as compared with the RAPA-untreated control) as expected from previous experiments. However, MIP-1β levels in the CCR5-null donor were only increased by 1.2-fold in the presence of RAPA.

### Example 9

Antiviral Activity of RAPA in PBMCs.

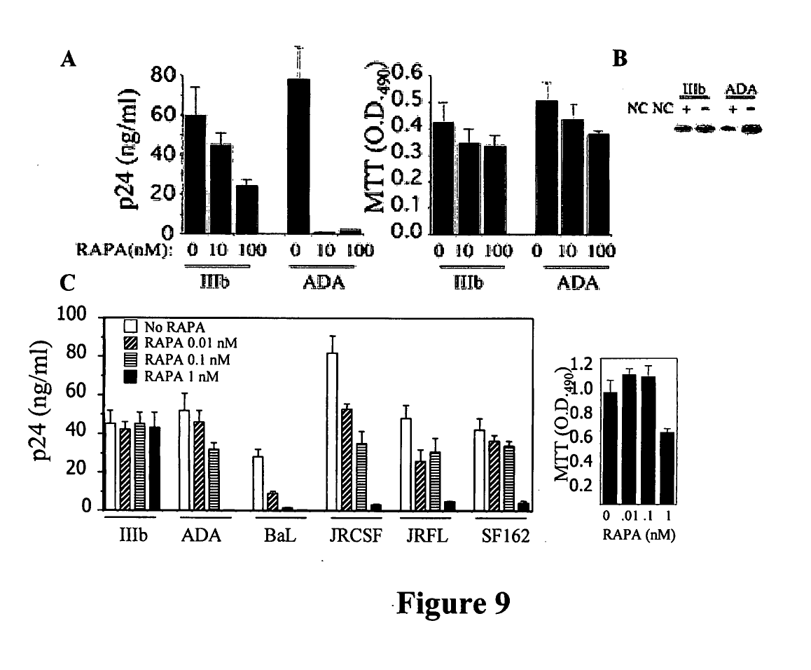

The antiviral activity of RAPA was assayed in PBMCs that had been cultured in the presence of RAPA for 7 days before infection. Cells were infected with the X4 HIV-1 IIIb and the R5 HIV-1 ADA strains. Infected cells were cultured in the presence of RAPA (same concentration as during pretreatment) for 7 additional days, during which time virus replication and cell viability were measured. In a total of seven different experiments using cells from different donors, the antiviral effect of RAPA was more potent against HV-1 ADA than against HIV-1 IlIb. At 10 nM RAPA, the average value of HIV-1 ADA inhibition in the seven experiments was 91% (range of 88-97%), whereas at 100 nM RAPA, HIV-1 ADA was inhibited by 94% (range of 92-99%). In contrast, 10 nM RAPA inhibited HIV-1 IIIb by 13.5% (range of 5-25%), and 100 nM RAPA inhibited HIV-1 IIIb by 32% (range 29-60%). Results obtained in one of the donors are shown in FIG. 9A.

To further demonstrate the disproportionate antiviral effect of RAPA on R5 versus X4 HIV-1 strains, the antiviral effect of the drug was next assessed by measuring viral DNA in cells shortly after infection. Donor PBMCs that had been cultured for 1 week in the presence (100 nM RAPA) or absence of drug were infected with DNase-treated stocks of R5 HIV-1 ADA or X4 HIV-1 IIIb. At 24 h after infection, cell lysates were prepared and amplified for HIV-1 DNA sequences by PCR. Amplified PCR products were detected by using a radiolabeled probe (FIG. 9B). Phosphoimager analyses of the radioactive signals indicated that HIV-1 IIIb DNA content was the same in the RAPA-treated and untreated cells. In contrast, HIV-1 ADA DNA content in the RAPA-treated cells was three times lower than in the untreated cells. Primer pairs specific for the β-actin gene indicated the same DNA input among samples (data not shown).

As the results obtained with HIV-1 IIIb and HIV-1 ADA suggested that RAPA exerted a more potent antiviral effect in R5 than in X4 HIV-1, the antiviral activities of low concentrations of RAPA (0.01, 0.1, and 1 nM) were next evaluated against a panel of five R5 strains of HIV-1 (FIG. 9C). At these concentrations of RAPA, antiviral activity was seen against R5 strains but not against HIV-1 IIIb. RAPA at 0.01 nM inhibited R5 HIV-1 by 10-64% depending on the strains, whereas 0.1 nM RAPA inhibited virus replication by 15-85%. At 1 nM RAPA, all R5 viruses were inhibited by >90%. Together, these results demonstrate that RAPA decreases the susceptibility of PBMCs to be infected by CCR5-using strains of HIV-1 while having little effect in CXCR4-using strains.

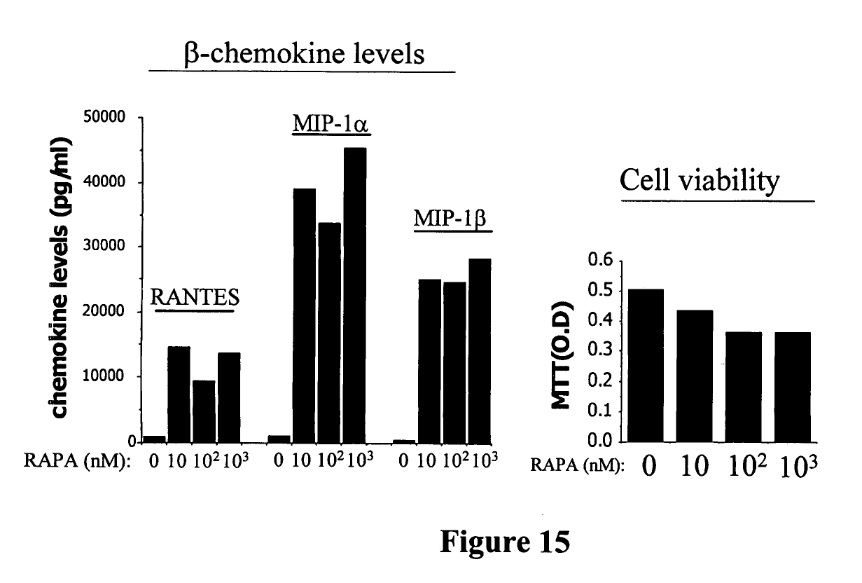

Further results are shown in FIGS. 15A and B wherein rapamycin disrupted signaling through the IL-2 receptor, leading to accumulation of lymphocytes in the G1 phase and thus an increase of chemokines. PBMCs from a healthy donor were cultured in the presence of IL-2 and Rapamycin. On day 7, chemokines content in the supernatant was measured by ELISA and cell viability was determined by the MTT assay.

### Example 10

Antiviral Activity of RAPA in Macrophages.

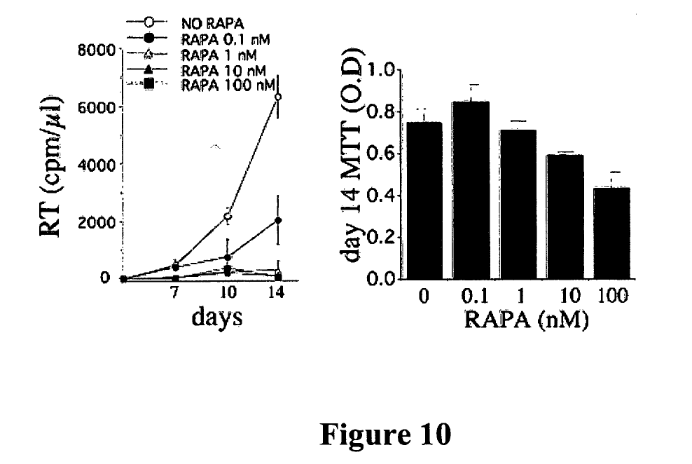

Donor monocytes were cultured for 5 days in the presence of RAPA. On day 5, cells were infected with HIV-1 ADA. Infected cells were cultured in the presence of RAPA for an additional 14 days. Virus production was measured on the culture supernatants on days 7, 10, and 14 after infection. Cell viability was measured by the MTT assay at the end of the experiment (FIG. 10). Over the course of the experiment, RAPA inhibited virus replication in a dose-dependent manner. On day 14, RAPA concentrations ranging 0.1-100 nM inhibited virus production by 70-95%. Cell viability at the end of experiment was reduced at RAPA concentrations >10 nM. In an additional experiment in which RAPA was used at 0.01 nM, the R5 viruses HIV-1 ADA and HIV-1 SF 162 were inhibited by 64% and 45%, respectively (data not shown).

In the above-described experiment, monocytes had been pretreated with RAPA during the 5-day differentiation period. To control for the possible interference of RAPA with the process of monocyte differentiation, a new infection experiment in which RAPA was not present during the 5-day monocyte differentiation period was designed. To this end, fresh monocytes were cultured for 5 days in the absence of RAPA. On day 5, cells were infected with HIV-1 ADA and then exposed to RAPA. Under these experimental conditions, two independent experiments using monocytes from two different donors indicated that 1 nM RAPA inhibited virus replication by ˜60% in one of the donors and by ˜80% in the other donors (virus production measured on day 14 after infection; data not shown).

Taken together, these results show that RAPA treatment of differentiating monocytes interferes with their ability to become susceptible targets for HIV infection and that RAPA also interferes with the ability of HIV to replicate in already differentiated macrophages.

### Example 11

RAPA Enhances the Antiviral Activity of the CCR5 Antagonist TAK-779.

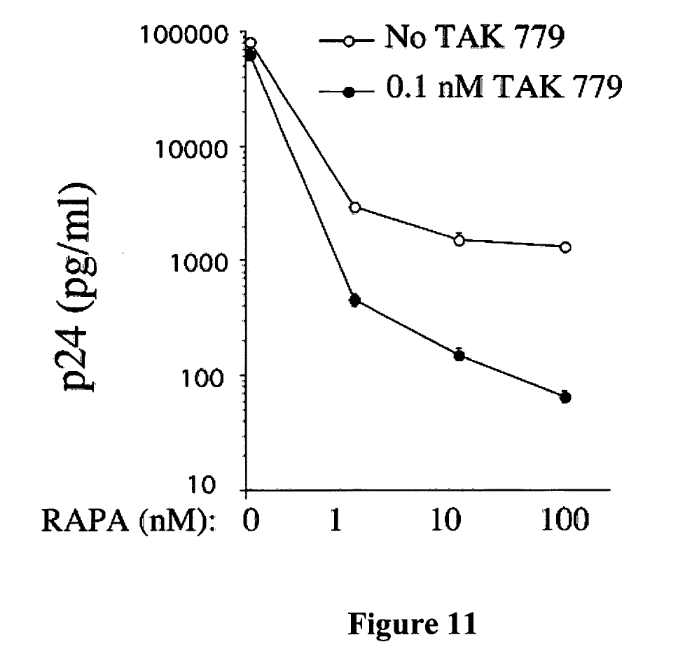

It was tested whether RAPA would increase the potency of a CCR5 antagonist drug. To test this hypothesis, donor PBMCs were cultured in IL-2 medium in the absence or presence of RAPA (1, 10, and 100 nM). After 7 days, cells were infected with HIV-1 ADA in the presence of 0.1 nM TAK-779, a concentration of drug showing little antiviral activity. Infected cells were cultured in the presence of RAPA (same concentration as during pretreatment) plus 0.1 nM TAK-779. Virus production was determined 7 days after infection (FIG. 11). In the absence of RAPA, 0.1 nM TAK-779 caused a 21% inhibition of virus replication. However, in the presence of 1 nM RAPA (a concentration of RAPA already exerting a potent antiviral effect), the antiviral effect due to TAK-779 increased from 21% to 74.5% virus inhibition. Similarly, the antiviral activity of TAK-779 was increased to 89% and 96% virus inhibition in the presence of 10 and 100 nM RAPA, respectively. Finally, RAPA is shown to enhance the antiviral activity of a CCR5 antagonist molecule. Also, our results indicate that low concentrations of the antagonist TAK-779 exert a more potent antiviral effect when added in the presence of RAPA. The TAK-779 concentration used did not affect cell viability (data not shown). These results suggest that the antiviral properties of a CCR5 antagonist drug are enhanced by RAPA thereby forming a synergistic combination.

The effects of RAPA on extracellular β-chemokine levels could help protect lymphocytes and macrophages against HIV-1 infection. The basis for these uses of the drug lies on its potent antiproliferative activity in cells. Although it may not seem appropriate to suggest the use of an immunosuppressant in HIV-infected individuals, it is important to point out that RAPA exerts a potent antiviral activity at concentrations lower than the ones used to cause immunosuppression in patients. In renal transplant recipients, a daily administration of 2 and 5 mg of RAPA results in therapeutic through levels of 9.3±4.4 nM and 18.9±8 nM, respectively [Rapamune package insert (2002), Wyeth]. In our studies, 0.01 and 0.1 nM RAPA inhibited the replication of some R5 strains of HIV-1 in PBMCs without affecting cell proliferation. RAPA concentrations of 1 nM had mild antiproliferative effects on cells and profoundly suppressed the replication of all R5 strains tested.

Our studies suggest that RAPA would be more effective in controlling the replication of R5 than X4 strains of HIV-1. In this regard, the therapeutic use of RAPA as a treatment of early HIV-1 disease (before appearance of X4 strains) may prove of value, particularly in light of current guidelines that advocate delayed initiation of antiretroviral therapy. Furthermore, the antiviral properties of RAPA could be especially relevant in geographical areas where subtype C HIV-1 is present, as these viruses use CCR5 as major coreceptor. Subtype C HIV-1 infections have risen in prevalence over the last decade, and they currently constitute the predominant subtype worldwide (Essex, M. 1999).

In summary, the ability of RAPA to augment extracellular levels of β-chemokines offers a new strategy with important implications for the treatment and prevention of HIV-1 infection. The synergic combination of RAPA and CCR5 antagonists may prove especially effective in controlling virus replication in patients.

### Example 12

Vitamin E (Alpha-Tocopherol) Increases Extracellular β-Chemokine Levels in Cultured PBMCs.

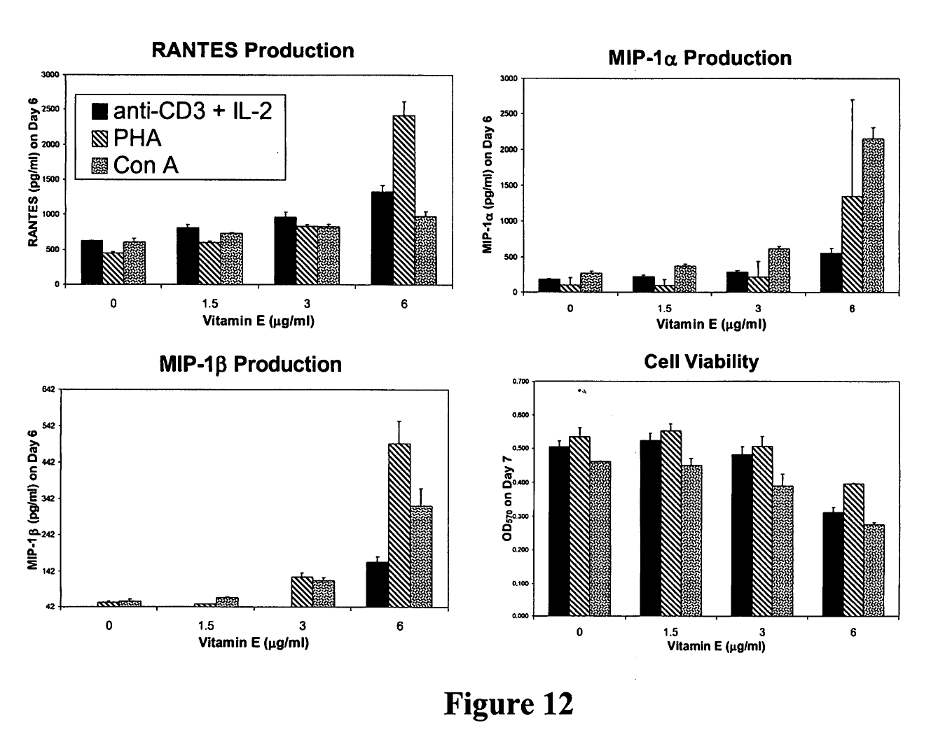

B-chemokines levels were measured in activated PBMCs cuftured in the presence of Vitamin E, an antioxidant reported to induce G1 cycle arrest. PBMCs from a healthy donor were activated under the presence of the indicated stimuli for 72 h. Activated cells were cultured in the presence of IL-2 and Vitamin E. Supernatants were evaluated for β-chemokine content on day 6 after addition of Vitamin E. Cell viability was measured by MTT assay. As was the case with other G1 agents evaluated, treatment of activated PBMCs with Vitamin E resulted in increased levels of β-chemokines as shown in FIG. 12.

### Example 13

Effects of Vitamin E on HIV-1 Production Upon Activation of Patient's Resting Cells.

In an additional set of experiments, the G1 phase arresting agent Vitamin E was assessed for its ability to inhibit HIV-1 production by lymphocytes isolated from the blood of HIV-1 infected individuals. Patients infected with HIV-1 interrupt antiretroviral therapy for multiple reasons including, amongst others, medication side effects, interrupted drug supply, acute illness and hospitalization. After treatment withdrawal a rapid rise in plasma viral loads occurs, which becomes detectable within 7-14 days (1). Latently infected, resting CD4 T cells serve as one possible source of rebounding virus upon treatment discontinuation. Treatment interruptions pose the risk of virus rebound, which can lead to the emergence of drug-resistant variants and to an increased risk of virus transmission.

HIV-1 gene expression on latently infected resting T cells is dependent on host transcriptional factors, such as NF-κB, that are induced on activated cells. In the present study we have evaluated the in vitro effect of adding Vitamin E (VE) to cultures of patients' resting CD4 T cells under conditions of cellular activation that promote virus expression.

Blood (50 ml) was drawn from 10 patients on antiretroviral therapy, whose viral load were <400 copies/ml and whose CD4 counts were >300/μl. Resting CD4 T cells were purified by negative selection using antibody-labeled magnetic beads (Dynal, Lake Success, N.Y.). Purified cells were activated by co-culture with γ-irradiated PBMCs from normal donors in the presence of 1 μg/ml anti-CD3 antibody (Coulter, Miami, Fla.) and 100 units/ml rhIL-2 (Roche, Indianapolis, Ind.). Cultures were set up in the absence or continuous presence of 5 μg/ml of VE (Vitamin E succinate, Sigma, St. Louis, Mo.), with medium replenishment every 3 or 4 days. Cultures were maintained for 14 days. Virus production was monitored by measuring p24 antigen (NEN, Boston, Mass.) in the culture supernatants on days 7 and 14. Cell viability was measured by trypan blue staining.

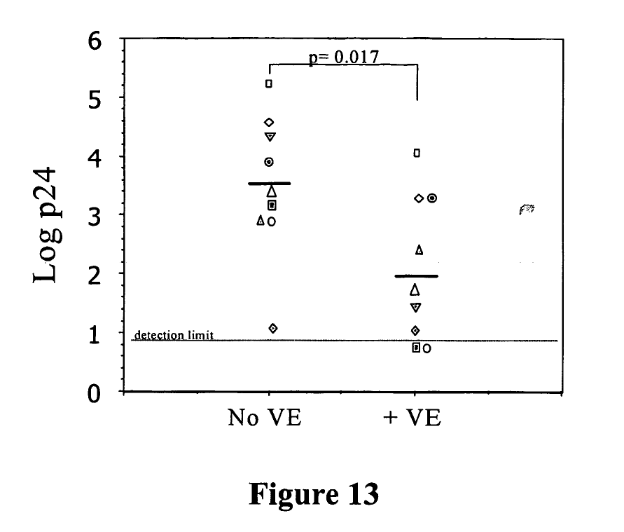

On day 14, virus isolation was positive in 9/10 untreated cultures and in 7/10 VE-containing cultures. Virus isolation was unsuccessful in one patient under either condition. FIG. 13 shows virus culture results obtained in the 9 remaining patients. Virus production was higher in the absence (mean Log p24=3.58) than in the presence of VE (mean Log p24=2.06), and this difference was statistically significant (two-tailed, paired t test, p=0.016). A similar pattern of antiviral results was obtained on day 7. Neither cell viability nor cell proliferation was affected by the concentration of VE used on days 7 or 14 (data not shown).

These in vitro results demonstrate that VE suppresses production of HIV-1 by patients' resting CD4+ T cells upon cellular activation. Further, the results demonstrate that the G1 cell cycle agent Vitamin E profoundly inhibits the in vitro production of HIV-1 by patient lymphocytes.

### Example 14

In vivo effects of Rapamycin on Expression of the Chemokine Receptor 5 (CCR5) and the Chemokines MIP1α, MIP-1β and RANTES in Healthy Adults

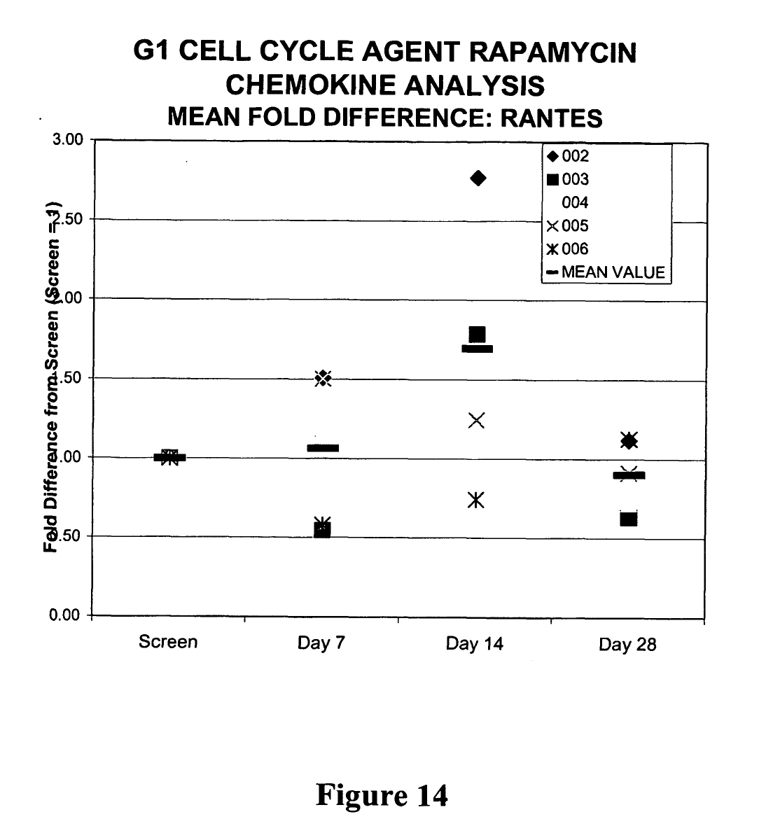

HIV-1 has been shown in most instances to use the chemokine receptor, CCR5, as a co-receptor for entry into macrophages and CD4 lymphocytes. The natural ligands for the CCR5 co-receptor are proteins called β-Chemokines. In an in vitro model, discussed above, it was demonstrated that Rapamycin markedly increased the activity of β-chemokine levels. To assess the activity of β-Chemokine production levels and its effects on CCR5 receptors in vivo an open-labeled, non-randomized observational trial was performed in which 5 healthy volunteers were given 2 mg/day of Rapamycin, following a 6 mg loading dose, for 14 days. Peripheral blood for determining β-Chemokines was obtained at the screening visit, days 7 and 14 of the Rapamycin dosing, and at day 28 (which was two weeks following the last dose of Rapamycin). All 5 subjects completed the study and Rapamycin was well-tolerated. There was an increase in β-Chemokine levels over baseline in all 5 subjects with Rapamycin therapy, which returned to baseline at day 28; the results of the RNA expression of CCR5 by CD4 lymphocytes are pending, as shown in Table I. FIG. 14 shows the results of all five subjects for increase of RANTES. Thus, altering the cell-cycle of peripheral blood mononuclear cells with a G1-specific agent, Rapamycin, resulted in the increased levels of β-Chemokines in healthy volunteers; and, this agent was well-tolerated.

TABLE I

| Patient | MIP-1β | RANTES |
|---|---|---|
| 002 | ↑ | ↑ |
| 003 | ↑ | ↑ |
| 004 | ↑ | ↑ |
| 005 | ↑ | ↑ |
| 006 | ↑ | ↓ |

### Example 15

Use of G1 cell cycle agents by HIV infected subjects resulted in a delay in viral rebound, allowing for lengthened cycles of “off” therapy, yet still maintain viral suppression.

Several studies have shown remarkable consistency in the kinetics of viral rebound once therapy has been stopped. The mean time to detectable viral load greater than 50 c/ml once therapy is withdrawn is 11 days. (Davey, et al. 1999)

The protocol used in the present invention expands on the NIH's concept of Cyclic Therapy. Cyclic therapy is a strategy in which patients can maintain viral suppression despite alternating between 7 days of taking ARV medications and 7 days of taking no medications. Compared to the NIH trail of short cycle of 7 days intermittently off therapy, in our trial, the G1 cycle agent HU is being used to maintain viral suppression during an extended off medication cycle of 14-28 days. The off period is followed by 7 days of combination antiretroviral therapy. This has allowed study participants to be on ⅔ fewer medications but still maintain viral control and immune reconstitution.

Four patients are enrolled in the trial. Data is available on 3 patients and is shown in the Table II below. Patients in this study have benefited from decreased drug related toxicities, relief from difficult financial sacrifices to stay on ARV's, and improved adherence to medications.

| | Baseline values at | | WEEKS OFF CONTINUOUS THERAPY | | | | | | | |
|---|---|---|---|---|---|---|---|---|---|---|
| | time of stopping | | (7 days on ARV's alternated by 14 days off ARV's but on HU) | | | | | | | |
| Patient | continuous therapy | | 3 wks | 6 wks | 9 wks | 12 wks | 15 wks | 18 wks | 21 wks | 24 wks |
| 1 | Viral load | 0 | 72 | 158 | 114 | 322 | 973 | 0 | | |
| | Copies/ml | | | | | | | | | |
| | Absolute | 377 | | | | | | 518 | | |
| | CD4 count | | | | | | | | | |
| 2 | Viral load | 0 | 0 | 0 | 0 | 0 | 0 | | | |
| | c/ml | | | | | | | | | |
| | Absolute | 1256 | | | | | 973 | | | |
| | CD4 count | | | | | | | | | |
| 3 | Viral load | 0 | 0 | 0 | 0 | | | | | |
| | c/ml | | | | | | | | | |
| | Absolute | 934 | | | 995 | | | | | |
| | CD4 count | | | | | | | | | |

As noted above, the first three patients have completed at least 3 cycles of treatment. All three patients demonstrated persistent viral suppression despite discontinuation of primary anti-retroviral drugs. This pilot study supports the conclusion that the use of a G1 cell cycle agent (i.e. HU) during periods where antiretroviral therapy is interrupted, will provide therapeutic benefits as reflected by persistent viral suppression. This is in direct contrast to state of art where viral rebound in 10,000-100,000 range would be expected by day 10-11 in patients who discontinued antiretroviral regimen without the continued use of a G1 cell cycle agent. Thus, these data support the clinical utility of the capacity of G1 cell cycle agents to augment the host ability to maintain viral control in the absence of primary combination anti-retroviral drug therapy and supports the clinical relevance of our in vitro findings that G1 cell cycle agents increase β-chemokines antiviral activity. 15

### REFERENCES

The contents of all cited references (including literature references, issued patents, published patent applications as cited throughout this application are hereby expressly incorporated by reference.
Adams, D. H. & Lloyd, A. R. (1997) Lancet 349, 490-495.
Baba, M., Imai, T., Yoshida, T. & Yoshie, O. (1996) Int. J. Cancer 66, 124-129.
Baba, M., Nishimura, O., Kanzaki, N., Okamoto, M., Sawada, H., Iizawa, Y., Shiraishi, M., Aramaki, Y., Okonogi, K., Ogawa, Y., et aL (1999) Proc. Natl. Acad Sci. USA 96, 5698-5703.
Bleul, C., Wu, L., Hoxie, J., Springer, T. & Mackay, C. (1997) Proc. Natl. Acad Sci. USA 94, 1925-1930.
Cocchi, F., DeVico, A. L., Yarchoan, R., Redfield, R. R., Cleghom, F., Blattner, W. A., Garzino-Demo, A., Colombini-Hatch, S., Margolis, D. D. & Gallo, R. C. (2000) Proc. Natl. Acad Sci. USA 97, 13812-13817.
Castillo, R. C., Arango-Jaramillo, S., John, R., Weinhold, K., Kanki, P., Carruth, L. & Schwartz, D. H. (2000) J. Infect. Dis. 181, 897-903.
Cocchi, F., DeVico, A., Garzino-Demo, A., Arya, S., Gallo, R. & Lusso, P. (1995) Science 270, 1811-1815.
Cossarizza, A., Ortolani, C., Mussini, C., Borghi, V., Guaraldi, G., Mongiardo, N., Bellesia, E., Francheschini, M. G., DeRienzo, B. & Francheschini, C. (1995) J. Infect. Dis. 172, 105-112.
Davey R, Bhat N, Yoder C, et al., Proc Natl Acad Sci USA (1999), 96: 15109-15114.
Dybul, M., Fauci, A., Barlett, J., Kaplan, J. & Pau, A. (2002) Ann. Intern. Med 137, 381-433.
Dybul, M., Chun, T W., Yoder, C., Proc Natl Acad Sci USA (2001), 10:1073.
Essex, M. (1999) Adv. Virus Res. 53, 71-88.
Ferbas, J., Giorgi, J. V., Amini, S., Grovit-Ferbas, K., Wiley, D. J., Detels, R. & Plaeger, S. (2000) J. Infect. Dis. 182, 1247-1250.
Gao, W. Y., Cara, A., Gallo, R. C. & Lori, F. (1993) Proc. Natl. Acad. Sci. USA 90, 8925-8928.
Gray, N., Detivaud, L., Doerig, C. & Meijer, L. (1 999) Curr. Med Chem. 6, 859-875.
Gualberto, A., Aldape, K., Kozakiewicz, K. & Tlsty, T. (1998) Proc. Natl. Acad. Sci. USA 95, 5166-5171.
Korin, Y. & Zack, J. (1998) J. Virol. 72, 3161-3168.
Koostra, N. A., Zwart, B. M. & Schuitemaker, H. (2000) J. Virol. 74, 1712-1717.
Levine, B., Mosca, J., Riley, J., Carroll, R. G., Vahey, M. T., Jagodzinski, L. L., Wagner, K. F., Mayers, D. L., Burke, D. S., Weislow, O. S., et al. (1996) Science 272, 1939-1943.
Lin, Y.-L., Mettling, C., Portales, P., Reynes, J., Clot, J. & Corbeau, P. (2002) Proc. Natl. Acad. Sci. USA 99, 15590-15595.
Liu, R., Paxton, W., Choe, S., Ceradini, D., Martin, S., Horuk, R., MacDonald, M., Stuhimann, H., Koup, R. & Landau, N. (1996) Cell 86, 367-377.
Lori, F., Malykh, A., Cara, A., Sun, D., Weinstein, J. N., Lisziewicz, J. & Gallo, R. C. (1994) Science 266, 801-805.
Noguchi, P. D. (1991) in Current Protocols in Immunology, eds. Coligan, J. E., Kruisbeek, A. M., Margulies, D. H., Shevach, E. M. & Strober, W. (Wiley, New York), pp. 5.7.1-5.7.6.
Paxton, W. A., Martin, S. R., Tse, D., O'Brien, J., Skumick, J., Van Devanter, N. L., Padain, N., Braun, J. F., Kotler, S. M., Wolinsky, S. M., et al. (1996) Nat. Med 2, 412-417.
Pemo, C. & Yarchoan, R. (1993) in Current Protocols in Immunology, eds. Coligan, J. E., Kruisbeek, A. M., Margulies, D. H., Shevach, E. M. & Strober, W. (Wiley, New York), pp. 12.4.1-12.4.11.
Ping, L. H., Nelson, J. A., Hoffman, I. F., Schock, J., Lamers, S. L., Goodman, M., Vernazza, P., Kazembe, P., Maida, M., Zimba, D., et al. (1999) J. Virol. 73, 6271-6281.
Poli, G. & Fauci, A. (1993) in Current Protocols in Immunology, eds. Coligan, J. E., Kruisbeek, A. M., Margulies, D. H., Shevach, E. M. & Strober, W. (Wiley, New York), pp. 12.3.1-12.3.7.
Strizki, J., Xu, S., Wagner, N., Wojcik, L., Liu, J., Hou, Y., Endres, M., Palani, A., Shapiro, S., Clader, J. W., et al. (2001) Proc. Natl. Acad Sci. USA 98, 12718-12723.
Tang A, Graham N, Semba R, Saah A., AIDS 1997, 11:613-620.
Trkola, A., Kuhmann, S., Strizki, J., Maxwell, E., Ketas, T., Morgan, T., Pugach, P., Xu, S., Wojcik, L., Tagat, J., et al. (2002) Proc. Natl. Acad Sci. USA 99, 395-400.
Weiss, A. (1999) in Fundamental Immunology, ed. Paul, W. E. (Lippincott, Philadelphia), pp. 413-415.
Zack, J., Arrigo, S., Weitsman, S., Go, A., Haislip, A. & Chen, I. (1990) Cell 61, 213-222.
Zagury, D., Lachgar, A., Chams, V., Fall, L. S., Bernard, J., Zagury, J. F., Bizzini, B., Gringeri, A., Santagostino, E., Rappaport, J., et al. (1998) Proc. Natl. Acad. Sci. USA 95, 3857-3861.

1. A pharmaceutical composition for increasing concentrations of chemokines to reduce entry of HIV virus into mononuclear cells through binding of chemokine binding receptors, the composition comprising at least one G1 phase arresting compound in an amount sufficient to increase concentrations of extracellular β-chemokines.

2. The pharmaceutical composition of claim 1, further comprising at least one antiviral agent.

3. The pharmaceutical composition of claim 1, wherein the G1 phase arresting compound is a member selected from the group consisting of sodium butyrate, aphidicolin, hydroxyurea (HU), olomoucine, roscovitine, tocopherols, tocotrienols, and rapamycin (RAPA).

4. The pharmaceutical composition of claim 2, wherein the antiviral agent is an HIV antiviral agent.

5. The pharmaceutical composition of claim 4, wherein the HIV antiviral agent is a nucleoside RT inhibitor, CCR5 inhibitors/antagonist, viral entry inhibitor or functional equivalent thereof.

6. The pharmaceutical composition of claim 2, wherein the antiviral agent is at least one member selected from the group consisting of: Zidovudine (ZDV, AZT), Lamivudine (3TC), Stavudine (d4T), Didanosine (ddl), Zalcitabine (ddC), Abacavir (ABC), Emirivine (FTC), Tenofovir (TDF), Delaviradine (DLV), Efavirenz (EFV), Nevirapine (NVP), Fuzeon (T-20), Saquinavir (SQV), Ritonavir (RTV), Indinavir (IDV), Nelfinavir (NFV), Amprenavir (APV), Lopinavir (LPV), Atazanavir, Combivir (ZDV/3TC), Kaletra (RTV/LPV), Trizivir (ZDV/3TC/ABC), SCH-C, SCH-D, PRO 140, TAK 779, TAK-220, RANTES analogs, AK602, UK-427, 857, monoclonal antibodies, NB-2, NB-64, T-649, T-1249, and functional analog thereof.

7. The pharmaceutical composition of claim 4, wherein the compound is administered orally, rectally, nasally, topically, vaginally or parenterally.

8. The pharmaceutical composition of claim 4, wherein the antiviral agent comprises tenofovir in combination with HU.

9. The pharmaceutical composition of claim 4, wherein the antiviral agent comprises tenofovir, 3TC and Efavirenz in combination with HU.

10. The pharmaceutical compositions of claim 2, wherein the composition is administered alone and in combination with the antiviral agent in a cyclic therapy program.

11. A method for inducing increased levels of anti-HIV β-chemokines in activated lymphocytes, the method comprising:
administering a composition comprising at least one G1 phase arresting agent in an effective amount to increase levels of anti-HIV β-chemokines, wherein the increased levels of anti-HIV β-chemokines bind to β-chemokine receptors thereby reducing viral entry of HIV.

12. The method according to claim 11, wherein the G1 phase arresting agent is a member selected from the group consisting of: sodium butyrate, aphidicolin, hydroxyurea (HU), olomoucine, roscovitine, tocopherols, tocotrienols, and rapamycin (RAPA).

13. The method according to claim 11, further comprising at least one antiviral agent.

14. The method according to claim 13, wherein the antiviral agent is an HIV antiviral agent.

15. The method according to claim 14, wherein the HIV antiviral agent is a nucleoside RT inhibitor, CCR5 inhibitors/antagonist, viral entry inhibitor or functional equivalent thereof.

16. The method according to claim 13, wherein the at least one antiviral agent is a member selected from the group consisting of: Zidovudine (ZDV, AZT), Lamivudine (3TC), Stavudine (d4T), Didanosine (ddl), Zalcitabine (ddC), Abacavir (ABC), Emirivine (FTC), Tenofovir (TDF), Delaviradine (DLV), Efavirenz (EFV), Nevirapine (NVP), Fuzeon (T-20), Saquinavir (SQV), Ritonavir (RTV), Indinavir (IDV), Nelfinavir (NFV), Amprenavir (APV), Lopinavir (LPV), Atazanavir, Combivir (ZDV/3TC), Kaletra (RTV/LPV), Trizivir (ZDV/3TC/ABC), SCH-C, SCH-D, PRO 140, TAK 779, TAK-220, RANTES analogs, AK602, UK-427, 857, monoclonal antibodies, NB-2, NB-64, T-649, T-1249, and functional analog thereof.

17. The method according to claim 13, wherein the compound is administered orally, rectally, nasally, topically, vaginally or parenterally.

18-19. (canceled)

20. The method according to claim 1, wherein the chemokine comprises MIP-1α, MIP-1β and RANTES.

21-36. (canceled)

37. A method of maintaining viral control of an HIV infection, the method comprising:
administering at least one antiviral agent in combination with at least one G1 phase arresting compound, wherein the G1 phase aresting compound is in a concentration sufficient to increase levels of β-chemokines.

38. The method according to claim 37, wherein the at least one antiviral agent and the at least one G1 phase arresting compound are administered concurrently.

39. The method according to claim 38, wherein the G1 phase arresting compound is a member selected from the group consisting of: sodium butyrate, aphidicolin, hydroxyurea (HU), olomoucine, roscovitine, tocopherols, tocotrienols, and rapamycin (RAPA).

40. The method according to claim 39, wherein the antiviral agent is at least one member selected from the group consisting of: Zidovudine (ZDV, AZT), Lamivudine (3TC), Stavudine (d4T), Didanosine (ddl), Zalcitabine (ddC), Abacavir (ABC), Emirivine (FTC), Tenofovir (TDF), Delaviradine (DLV), Efavirenz (EFV), Nevirapine (NVP), Fuzeon (T-20), Saquinavir (SQV), Ritonavir (RTV), Indinavir (IDV), Nelfinavir (NFV), Amprenavir (APV), Lopinavir (LPV), Atazanavir, Combivir (ZDV/3TC), Kaletra (RTV/LPV), Trizivir (ZDV/3TC/ABC), SCH-C, SCH-D, PRO 140, TAK 779, TAK-220, RANTES analogs, AK602, UK-427, 857, monoclonal antibodies, NB-2, NB-64, T-649, T-1249, and functional analog thereof.

41. The method according to claim 41, wherein the G1 phase arresting agent is HU.

42. The method according to claim 41, wherein the G1 phase arresting agent is rapamycin.

43. A therapeutically effective method to inhibit replication of HIV in a HIV infected subject, the method comprising:
a) administering at least one G1 phase arresting agent in a concentration sufficient to increase concentration of extracellular β-chemokines for a first predetermined time period; and
b) administering the G1 phase agent with at least one antiviral agent, for a second predetermined time period, wherein the first and second time periods are sequential in a cyclic schedule.

44. The therapeutically effective method according to claim 43, wherein the G1 phase arresting agent is a member selected from the group consisting of: sodium butyrate, aphidicolin, hydroxyurea (HU), olomoucine, roscovitine, tocopherols, tocotrienols, and rapamycin (RAPA).

45. The therapeutically effective method according to claim 43, wherein the antiviral agent is a nucleoside RT inhibitor, CCR5 inhibitors/antagonist, viral entry inhibitor or functional equivalent thereof.

46. The therapeutically effective method according to claim 43, wherein the antiviral agent is at least one member selected from the group consisting of: Zidovudine (ZDV, AZT), Lamivudine (3TC), Stavudine (d4T), Didanosine (ddl), Zalcitabine (ddC), Abacavir (ABC), Emirivine (FTC), Tenofovir (TDF), Delaviradine (DLV), Efavirenz (EFV), Nevirapine (NVP), Fuzeon (T-20), Saquinavir (SQV), Ritonavir (RTV), Indinavir (IDV), Nelfinavir (NFV), Amprenavir (APV), Lopinavir (LPV), Atazanavir, Combivir (ZDV/3TC), Kaletra (RTV/LPV), Trizivir (ZDV/3TC/ABC), SCH-C, SCH-D, PRO 140, TAK 779, TAK-220, RANTES analogs, AK602, UK-427, 857, monoclonal antibodies, NB-2, NB-64, T-649, T-1249, and functional analog thereof.

47. The therapeutic method according to claim 43, wherein the cyclic schedule comprises:
a) administering a combination of at least one antiviral agent and at least one G1 phase arresting agent to the HIV infected subject for a predetermined first time period;
b) administering the at least one G1 phase arresting compound to the HIV infected subject for a second predetermined time period;
c) administering the combination of the antiviral agent and G1 phase arresting agent to the HIV infected subject for a predetermined third time period, which is less than the first period;
d) administering the G1 phase arresting compound to the HIV infected subject for a fourth predetermined time period which is less than the second time period; and
e) maintaining the cyclic schedule of steps c and d until replication of the HIV increases to a predetermined level.

48-54. (canceled)

55. The method according to claim 11, wherein the chemokine comprises MIP-1α, MIP-1β and RANTES.

56. The method according to claim 11, wherein the chemokine receptor is CCR5.

57. The method according to claim 37, wherein the chemokine comprises MIP-1α, MIP-1β and RANTES.

58. The method according to claim 37, wherein the chemokine receptor is CCR5.

59. The method according to claim 43, wherein the chemokine comprises MIP-1α, MIP-1β and RANTES.

60. The method according to claim 43, wherein the chemokine receptor is CCR5.
